(19) United States
(12) Patent Application Publication
(10) Pub. No.: US 2023/0105892 A1
(43) Pub. Date: Apr. 6, 2023

(54) DEVICE AND METHOD FOR URINE ANALYSIS

(71) Applicant: WITHINGS, ISSY LES MOULINEAUX (FR)

(72) Inventors: Thibaut BARBEDETTE, ISSY LES MOULINEAUX (FR); Marine KIRAT, ISSY LES MOULINEAUX (FR); Jonas LOY, ISSY LES MOULINEAUX (FR); Benoît TUCOULAT, ISSY LES MOULINEAUX (FR); Julius DEWAVRIN, ISSY LES MOULINEAUX (FR)

(21) Appl. No.: 17/799,618

(22) PCT Filed: Mar. 3, 2021
(86) PCT No.: PCT/EP2021/055302

§ 371 Date: Aug. 12, 2022

(30) Foreign Application Priority Data
Mar. 3, 2020 (FR) FR2002124

(51) Int. Cl. A61B 5/20; A61B 5/00; A61B 10/00

(52) U.S. Cl. CPC A61B 5/207; A61B 5/6891; A61B 10/007; A61B 2010/0006

(57) ABSTRACT

A urine analysis device for positioning within a toilet includes a test assembly having at least one rotatable holder including a plurality of test strips attached to the rotatable holder; an injector configured to inject a controlled volume of urine onto at least one of the test strips; and an analysis system configured to detect a result of urine injection onto the test strip.

### FIELD OF THE INVENTION

The present disclosure is in the field of urine analysis devices to be positioned inside a toilet. The present disclosure also relates to a method for analyzing urine being received in a toilet.

### BACKGROUND OF THE DISCLOSURE

Many biological parameters are reflected in the urine of an individual. For example, health problems such as urinary tract infection, diabetes or kidney failure can be detected from a urine sample. The urine sample can also reflect the quality of a diet, identify a fertile period or pregnancy and detect drug or tobacco use. It is then interesting to monitor various biological parameters periodically.

It is known to offer devices installed in toilets with a urine analysis function. These devices are capable of taking urine samples from the toilet and analyzing them to determine the level of a biological parameter.

A urine analysis device to be attached to a toilet rim is known from US20180188231. This device allows an analysis with a field effect transistor.

In US20170284925 and U.S. Ser. No. 10/383,606, devices with test strips running past an analysis section are proposed.

However, such devices are bulky. In particular, they require a storage area for new and used test strips. These devices must then be positioned largely outside the toilet or must be integrated into the toilet.

Furthermore, such devices are not flexible. It appears particularly difficult to refill new test strips and to remove used strips. It is also difficult to perform tests that require multiple types of strips.

Therefore, there is a need for a urine analysis device that does not have the drawbacks of the prior art.

### SUMMARY OF THE DISCLOSURE

The present description aims at proposing one or more solutions solving at least some of the above-mentioned drawbacks.

In one embodiment, there is provided a urine analysis device to be positioned within a toilet comprising a test assembly including:
at least one rotatable holder comprising a plurality of test strips attached to the rotatable holder;
an injector configured to inject a controlled volume of urine onto at least one of the test strips;
an analysis system configured to detect a result of urine injection onto the test strip.

Thus, advantageously, the urine analysis device is compact enough to be positioned entirely inside the toilet. It can then be discrete and easily installed and removed. It can also be adapted to any type of toilet. The use of a rotatable holder on which the strips are fixed allows a plurality of analyses to be carried out in a very simple way. The use of such a rotatable holder allows to reduce the size of the device for a given number of tests. The rotatable holder can also be replaced. The device is thus modular and versatile.

The term ‘rotatable holder’ is used here to mean that the holder is a part that is rotatably mounted on a base part. The urine analysis device without the rotatable holder is referred to as a station (the base part is thus part of the station). The term cartridge will also be used for the rotatable holder, as the latter is a replaceable consumable. The station and the cartridge are two separate entities that can be manufactured and sold independently of each other.

In one embodiment, there is also provided a cartridge for a urine analysis device (which will therefore be interchangeably referred to as a cartridge or a rotatable holder), the cartridge being configured to be rotatably mounted on the station (e.g., a base of the urine device) of the urine analysis device as described above, the cartridge comprising a plurality of test strips, attached to the cartridge.

In one embodiment, there is also provided a station fora urine analysis device as described above. The station is configured to receive a rotatably mounted cartridge comprising a plurality of test strips. The station typically comprises an injector configured to inject a controlled volume of urine onto at least one of the test strips and an analysis system configured to detect a urine injection result on the test strip.

In one embodiment, there is also provided a kit comprising a station as disclosed above and at least one cartridge as disclosed above. In particular, a kit may include two cartridges and the two cartridges may have a different strip configuration from each other (to measure different compounds).

The features outlined in the following paragraphs can optionally be implemented. They can be implemented independently of each other or in combination with each other.

The or each rotatable holder can be configured to rotate a test strip and selectively present it in front of the injector and/or the analysis system. This reduces the number of moving parts in the device. It is also possible to select a specific type of strip to perform an analysis. The device is versatile and modular.

The or each rotatable holder may be configured to rotate in both clockwise and counterclockwise directions. Thus, the test strip can be presented in front of the injector and/or analysis system along the shortest path. This arrangement also allows for a great deal of flexibility in the design of the device, particularly by reducing constraints on the positioning of the analyzer system or injector relative to the rotatable holder.

The or each rotatable holder may have housings that receive one or more test strips. Thus, the test strips are held in the rotatable holder. Each housing may separate one or more test strips from adjacent test strips, thereby improving the conservation of the strips and the accuracy of the analysis. The strips are secured in their housing so that they will not move from the housing during normal use of the rotatable holder.

The housings can be positioned along a circle or a portion of a circle, equidistant from the axis of rotation of the rotatable holder. In particular, the housings are arranged parallel to each other, and, more specifically, parallel to the axis of rotation of the rotatable holder. The rotatable holder is then essentially invariant by increments of rotation (except for the type of strips).

The housings can be closed by at least one lid, for example transparent or translucent. Thus, reagents contained in the test strips are preserved before analysis. They can be easily analyzed by the analysis system, especially by optical analysis.

The rotatable holder can be cylindrical, and the housings can be arranged on an outer wall of the rotatable holder. This configuration maximizes the number of test strips received in the housings of the rotatable holder. A large number of analyses can be performed without the need to reload the device with test strips.

In one embodiment, the housings can be provided on an inner wall of the cylindrical rotatable holder. This arrangement allows great flexibility in the design of the device, in particular by reducing the constraints on the positioning of the analysis system or the injector with respect to the rotatable holder.

Furthermore, in this arrangement, the injector and/or at least a portion of the analysis system can be arranged in a radially inner area of the rotatable holder. This maximizes the diameter of the rotatable holder without requiring an increase in the size of the case. This maximizes the number of test strips that can be accommodated and thus the number of analyses available.

The injector may include a movable syringe configured to move toward or away from a test strip. For this purpose, the syringe may be arranged on a linear motor. The linear motor is mounted on the station.

The strips can be positioned in a circle or a portion of a circle, equidistant from the axis of rotation of the rotatable holder. In particular, the strips can be arranged parallel to each other and, more specifically, parallel to the axis of rotation of the rotatable holder, which allows a high number of strips to be arranged per angular unit.

The rotatable holder may include a separator comprising the housings, the separator being made of a flexible material, such as elastomer, and a receptacle receiving the separator. The test strips can be easily mounted in the separator, which can then be received in the receptacle to form the rotatable holder. The construction of the rotatable holder is facilitated. The receptacle may typically include an annular portion and a cylindrical portion, radially external to the annular portion. The separator may be bonded to the receptacle, and more specifically to the cylindrical portion.

The strips and the separator on which the strips are mounted are called a measurement band. The measurement band can be manufactured independently of the receptacle and then mounted on the receptacle.

The separator may include holes opening into the housings, and the receptacle may include a transparent cylindrical portion (or having transparent areas) in contact with the separator, so that the analysis system detects an analysis result by transmission of light through the cylindrical portion and the holes in the separator. Light can pass through the rotatable holder via the test strips. The holes can guide the light to areas of interest on the test strips.

The rotatable holder may include one or more markers, the device may include a sensor configured to detect the markers. Thus, the device can accurately control the angular position of the rotatable holder.

The markers can be optical markers, for example lines or barcodes. The sensor can then perform an optical analysis to establish the position of the rotatable holder. In addition, the optical sensor can identify a type of test strip. The sensor can also be the same as the sensor in the analysis system. This reduces the complexity of the device by reducing the number of components used in its operation.

In one example, the markers may be holes in the separator that open into the housings. The sensor may be the same as the sensor in the analysis system.

In one example, the markers may include a cam, such as a cam mounted on the rotatable holder, which is configured to cooperate with a follower, mounted on the station. The cam and follower provide information about the angular position of the cartridge in the station. The cam typically comprises a succession or alternation of highs and lows on a periphery of the rotatable holder (for example on an outer periphery, such as that of the receptacle), the highs and lows being arranged so that each housing (and thus each strip) is radially aligned with a low (respectively a high). A discontinuity in the cam, identifiable by the follower, at a given position also defines an angular zero.

The injector may include urine monitoring means, the urine monitoring means comprising: a sleeve defining an internal cavity through which urine may flow; a first conductive electrode and a second conductive electrode extending through an outer wall of the sleeve to open into the cavity so as to be responsive to urine received in the cavity, the first electrode being spaced from the second electrode to delineate a reference volume of the cavity. A volume of urine can then be accurately measured. The repeatability of injecting urine onto a test strip is improved.

The urine monitoring means may include a third conductive electrode, disposed between one end of the sleeve and the first conductive electrode to measure a flow rate of urine flowing through the cavity. The measurement of the urine flow rate may provide the activation time of a pump to inject the controlled volume of urine onto the test strip. The need for pump calibration is reduced.

The injector can be configured to inject between 1 microliter and 20 microliters of urine onto a test strip, for example between 2 microliters and 4 microliters. Thus, the injector injects enough urine onto a test strip to perform a conclusive analysis.

The analysis system may comprise: at least one light source (e.g., at least one LED), disposed on one side of the rotatable holder, the at least one light source emitting light toward the rotatable holder, and, a sensor disposed on the other side of the rotatable holder, the sensor facing the at least one light source so as to detect light passing through the rotatable holder. The analysis system is then adapted to perform a light transmission analysis through the rotatable holder.

The station may include a motor configured to rotate the rotatable holder.

A case may enclose the test assembly, the case being configured to be positioned entirely within a toilet, in particular within a toilet bowl, against an interior wall of said bowl. The case then protects the test assembly from the outside. The device has no moving parts outside the case. Therefore, the urine analysis device is unobtrusive. The case is also free of stagnation points where urine could stagnate. The urine analysis device is hygienic. The case also has no moving parts outside the case, and therefore no swivel joints, which are problematic in this environment.

The rotatable holder can be removably arranged in the case (or more generally in the station). Thus, a user can remove the rotatable holder to replace it, for example to refill the device with test strips or to change the type of test strips.

The case may have a front face for receiving a stream of urine directly from a user urinating while seated on the toilet, a rear face opposite the front face, and a collection port, disposed on the front face or on the back face. The collection port may also be located at a boundary between the front and rear faces. Thus, advantageously, the urine analysis device can collect urine through the collection port directly as it flows on the case. A user does not need to worry about the position of the case when urinating in the toilet.

The case may include a drain port configured to drain urine. Thus, excess collected urine can be purged from the previously collected urine analysis device. A subsequent urine collection is then not contaminated by a previous collection. Several consecutive urine collections can be performed.

The case can have a general circular pebble shape. Thus, the shape of the case is defined by curved surfaces. Urine can flow down the entire case without stalling or forming air bubbles.

The urine analysis device may include a sensor for the presence of urine, in the vicinity of the collection port, the sensor being configured to detect the presence of urine, for example the sensor being a temperature sensor. Thus, the urine analysis device may trigger an analysis when urine is detected on the case.

The urine presence sensor can be a temperature sensor. The temperature sensor can distinguish between urine and water being detected on the case. In addition, the temperature sensor can detect a fertile period. So the temperature sensor can both detect the presence of urine and perform an analysis. The number of components of the urine analysis device is reduced.

The urine analysis device may include a module for communication, for example wireless communication, with a remote device and/or a server and/or a smartphone. Thus, the urine analysis device may be controlled to initiate an analysis. The urine analysis device may analyze and transmit one or more analysis results.

The remote device can include a button. Then, an analysis can be triggered when a user presses the button.

The button can be equipped with a biometric sensor. Then the user can be identified and the analysis will only start if a user is identified. An analysis can be adapted to the identified user.

In one embodiment, a measurement band for a urine analysis device cartridge is also provided. This band may typically be part of the cartridge (also referred to as a rotatable holder) described above. The band may extend in a longitudinal direction and include a plurality of strips arranged parallel to each other. The strips typically each have a maximum dimension of less than 30 mm, or even 20 mm, or even 15 mm. They can be arranged perpendicular to the longitudinal direction, so that the strip can comprise a large number of strips and still be windable.

The features outlined in the following paragraphs can optionally be implemented. They can be implemented independently of each other or in combination with each other.

The strips can be rectangular in shape, each with a width of between 0.5 and 3 mm and a length of between 10 and 15 mm.

The band may include a separator, the separator having housings for receiving the strips. The separator may be made of a flexible material, such as elastomer.

The band may include at least 50 strips or at least 100 strips.

The band can be flexible so that it can be wound in a circle or a circular arc.

The separator comprises holes opening into the housing.

Each housing can be closed by a lid, for example a transparent lid. The lid can be continuous to facilitate the assembly process.

The band may have a first side comprising the housings and a second side, opposite the first side. The second side may include at least one protrusion or recess, configured to engage with a complementary recess or protrusion of the cartridge receptacle.

Each housing may have two side walls perpendicular to the longitudinal direction. In this way, the two walls are parallel to each other when the measurement band is laid flat, but gradually move towards each other as the housing slopes inward when the band is wound (i.e., when the longitudinal axis of the band is arranged in a circle and the housings are positioned radially inwards, not radially outwards). The strips are then trapped in their housing by the sloping walls.

According to another aspect, a method of urine analysis using the urine analysis device is proposed, comprising:
positioning a test strip in front of the injector by rotation of the rotatable holder;
injecting a controlled volume of urine onto the test strip;
positioning a test strip in front of the analysis system by rotation of the rotatable holder;
analyzing the test strip to establish an analysis result, for example by optical analysis.

The features outlined in the following paragraphs can optionally be implemented. They can be implemented independently of each other or in combination with each other.

The method may include measuring a reference volume, the reference volume comprising the controlled volume of urine to be injected onto the test strip. The reference volume may also include a pre-injection volume. The pre-injection volume may allow for the expulsion of air present in the injector that could impact the volume of urine actually injected onto the test strip.

Analyzing the test strip may include a step of transmitting light through the rotatable holder. A light transmission analysis is performed.

Injecting the controlled volume of urine can be done in two steps. The time of absorption and migration of urine on the test strip is thus taken into account.

The method can be triggered by a previous interaction step with a user. The method can be triggered by pressing a button. The method can be initiated by detecting a user in the vicinity of the toilet. The method can also be initiated upon detection of urine on the urine analysis device.

A user can select an analysis. For example, a user can choose the type of analysis he wants to perform.

The analysis result can be transmitted to a user's smartphone and/or a remote server.

### BRIEF DESCRIPTION OF THE DRAWINGS

Further features, details and advantages will become apparent from the detailed description below, and from an analysis of the attached drawings, in which:

FIG. 1 shows a schematic cross-section of a toilet equipped with a urine analysis device in the sense of the invention.

FIG. 2 represents schematically a detail of FIG. 1.

FIG. 3 illustrates a perspective view of a first case, according to a first embodiment, that can be implemented in the urine analysis device of FIG. 1.

FIG. 4 illustrates an exploded perspective view of the first case in FIG. 3 and a test assembly that can be implemented in the urine analysis device in FIG. 1.

FIG. 5 illustrates a partially exploded perspective view of a second case, according to a second embodiment, that can be implemented in the urine analysis device of FIG. 1.

FIG. 6 illustrates a detail of a third case, according to a third embodiment, that can be implemented in the urine analysis device of FIG. 1.

FIG. 7 shows another perspective view of the first case in FIG. 3.

FIG. 8 illustrates a perspective view of the test assembly visible in FIG. 4, from an alternate viewpoint on the opposite side.

FIG. 9 illustrates an exploded view of a first example of a subassembly that can be implemented in the test assembly.

FIG. 10 shows a cross-sectional view of a detail of the subassembly of FIG. 9.

FIG. 11 illustrates an exploded view of a second example of a subassembly that can be implemented in the test assembly.

FIG. 12 illustrates a perspective view of a third example of a subassembly that can be implemented in the test assembly.

FIG. 13 shows a perspective view of another assembly that can be implemented in the test assembly.

FIG. 14 illustrates a test strip that can be used in the urine analysis device.

FIG. 15 shows a block diagram of the test assembly in FIG. 4.

FIG. 16 illustrates a flowchart of a first method of using the urine analysis device.

FIG. 17 illustrates a flowchart of a second method of using the urine analysis device.

FIG. 18 shows a fourth example of a case that can be implemented in the urine analysis device mounted in a toilet.

FIG. 19 shows an exploded view of the fourth case example in FIG. 18.

FIG. 20 shows a top view of the interior of the case in FIG. 18.

FIG. 21 illustrates a perspective view of a rotatable holder comprising a cam, according to one embodiment.

FIG. 22 broken down into two figures FIG. 22a and FIG. 22b, illustrates a top view of a blocking/counting mechanism, according to one embodiment,

FIG. 23 illustrates a perspective view of a blocking/counting mechanism, according to one embodiment.

FIG. 24 illustrates a perspective view of a fourth example of a subassembly that can be implemented in the test assembly, focused on a rotatable holder.

FIG. 25 illustrates a partial view of a first side of a measurement band, according to one embodiment,

FIG. 26 shows a partial view of a second side of a measurement band, according to one embodiment,

FIG. 27 illustrates a complete perspective view of a measurement band, with magnification, according to one embodiment.

FIG. 28 illustrates a zoomed-in view of the rotatable holder, according to an embodiment.

FIG. 29 illustrates a side view of a urine control means, according to one embodiment.

FIG. 30 illustrates a cross-sectional view of an analysis system, according to an embodiment

FIG. 31 illustrates a top view of electronic components that can control the test assembly, according to one embodiment.

### DETAILED DESCRIPTION OF THE EMBODIMENTS

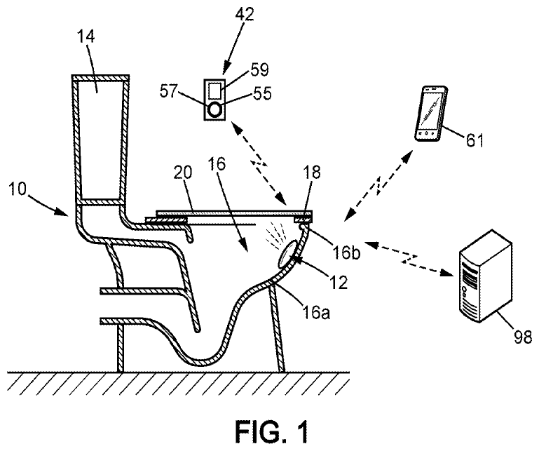

FIG. 1 illustrates a toilet 10 equipped with a urine analysis device 12. In a known manner, the toilet comprises a water tank 14, a bowl 16, a seat 18 and a lid 20. The urine analysis device is arranged on an inner wall 16a of the toilet bowl 16. Advantageously, the urine analysis device 12 is entirely received in the toilet bowl. Indeed, the urine analysis device is discrete.

The urine analysis device 12 is positioned in the path of a urine stream secreted by a user. The urine analysis device 12 receives a urine stream when a user urinates while sitting in the toilet. The position of the urine analysis device is then suitable for any type of user, male or female, regardless of age. The user can then urinate in the toilet without worrying about the position of the urine analysis device.

Here, the urine analysis device 12 is also positioned in the path of a flush from the cistern 14. Thus, the urine analysis device can be flushed when the toilet is flushed. The urine analysis device is hygienic.

The urine analysis device 12 comprises a case 22 enclosing a test assembly 24. The test assembly 24 is intended to analyze urine being received in the urine analysis device 12.

The case 22 is removably arranged in the toilet bowl 16. The analysis device can then be removed or repositioned in the toilet. The urine analysis device is discrete. In addition, the urine analysis device can be removed to recharge a battery 94 or a rotatable holder 44 of the test assembly 24.

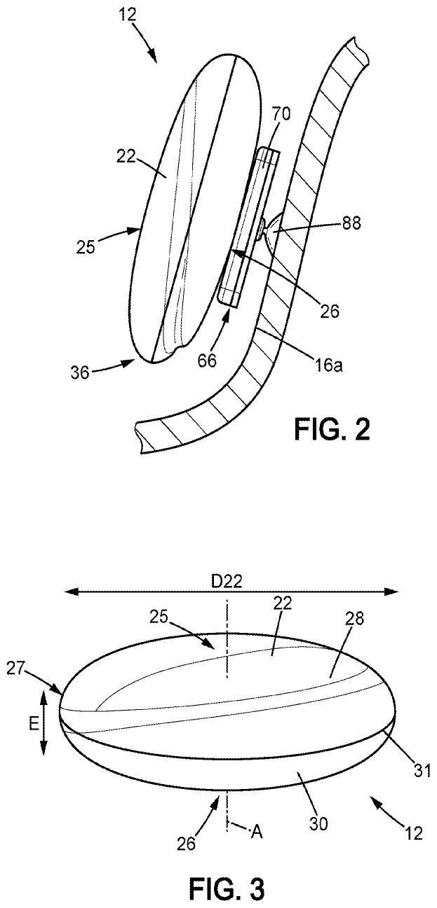

In the example shown in FIG. 2, the case 22 is arranged on the toilet wall 16a. The case is positioned by a fastener 66. The fastener 66 comprises a suction cup 88 intended to cooperate with the wall 16a of the toilet and magnets 70. The magnets are intended to cooperate with magnets 23 (or parts of ferromagnetic material) arranged inside the case. This configuration allows the case to be easily removed or repositioned in the toilet.

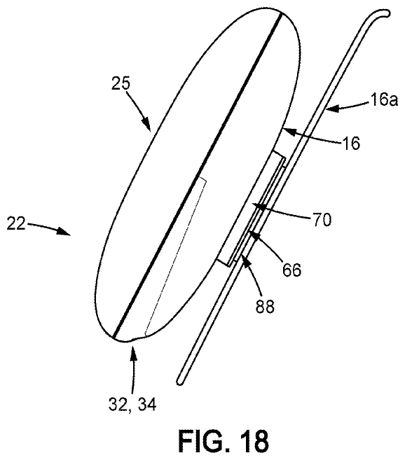

In the example shown in FIG. 18, the fastener 66 is further circular in shape. The fastener 66 fits into a complementary housing provided on the case 22. The housing is provided at battery charging connectors 94 of the urine analysis device 12. Thus, the charging connectors can be protected from the water of the toilet 10.

Mechanical aids, including indentations, may be provided on the fastener 66. The mechanical aids may facilitate the positioning of the case 22 when inserted into the toilet 10. The proper positioning of the case 22 in the toilet 10 ensures the proper functioning of the urine analysis device 12.

The fastener 66 may also include a ball-and-socket connection. The ball-and-socket connection allows the case 22 to be oriented to increase the likelihood of contact with urine being received in the toilet bowl 16.

Alternatively, the fastener 66 may be a hook mounted to a rim 16b of the toilet bowl.

General Characteristics of the Case

The case 22 has an outer shape of a circular pebble. In other words, the case has a flattened spheroid shape. An axis A is the median axis of the case. The case has a front face 25 and a rear face 26, substantially normal to the axis A. Thus, urine can be collected directly from the faces 25, 26 of the case. The case serves as a urine collector.

The front face 25 faces the inside of the toilet bowl 16. The front face 25 is then intended to receive urine when the user urinates while sitting on the toilet. The rear face 26 faces the inner wall 16a of the toilet bowl 16. The front face 25 and the rear face 26 are connected by curved edges 27. Thus, the outer surface of the case 22, consisting of the front face 25, the rear face 26 and the curved edges, is defined by curved lines, forming a generally convex object. The case has no ridges. Urine can run down the entire outer surface of the case without coming off the case or forming air bubbles, which can compromise a urine analysis.

The outer surface of the case 22 may further be white or light colored. The color of the outer surface may be similar to that of the toilet, increasing the discretion of the device.

In one embodiment, the case 22 has a diameter D22, measured in the direction normal to the axis A, of between 50 mm and 150 mm, for example near 100 mm. The case 22 also has a thickness E, measured in the direction of the axis A, of between 15 mm and 50 mm, for example close to 30 mm. Thus, the case is sufficiently compact to be completely received in the toilet bowl. The urine analysis device is discreet. In addition, the case is large enough to systematically come into contact with urine being received in the toilet bowl. The user can then urinate in the toilet without worrying about the urine analysis device, or alternatively aiming it roughly.

The outer surface of the case is smooth. Thus, the urine stream coming into contact with the case clings to and spreads over the outer surfaces of the case. In one embodiment, the case is made of a hydrophilic material. For example, the case may be one of: a ceramic, a polyamide (PA), a silicone or a hydrophilic polymer. The outer surface of the case may also be treated with a hydrophilic surface treatment, for example acuWet® from Aculon, a hydrophilic polymer, or Pebax® from Arkema.

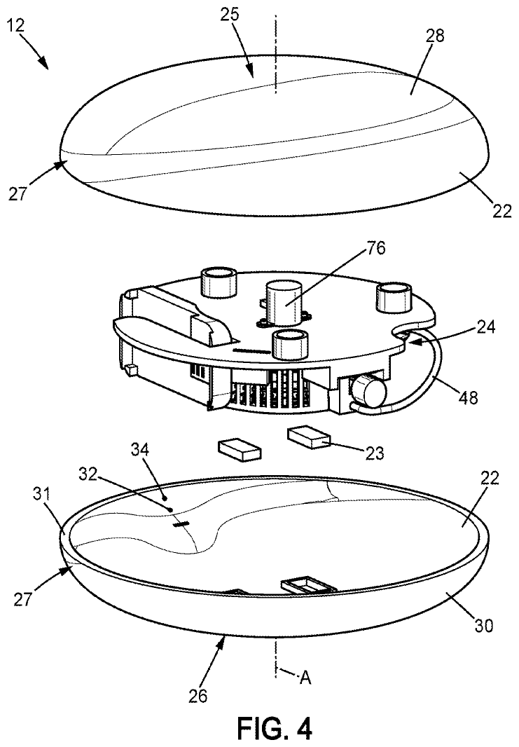

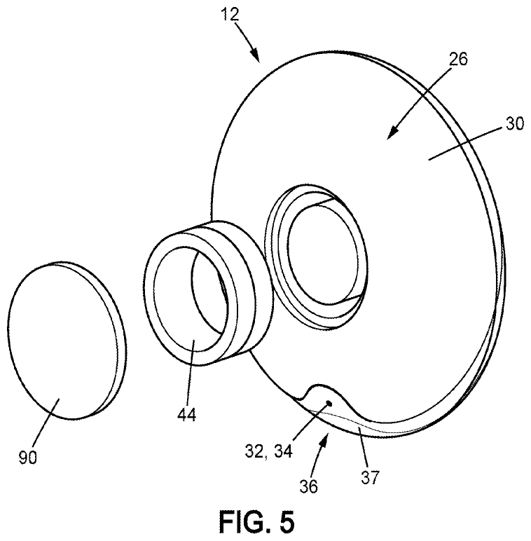

As more visible in FIG. 4, according to a particular embodiment, the case 22 is formed as an assembly of two half-shells and here consists of a front shell 28 and a rear shell 30. The front shell and the rear shell form a joint 31 of the case, in a plane normal to the axis A. The assembly of the urine analysis device is facilitated when the case consists of the front shell and the rear shell.

The front shell 28 and the rear shell 30 are joined to maintain the outer surface of the case defined by curved lines. Thus, the seam 31 between the front and rear shells allows urine runoff between the front and rear faces. The impact of the joint on urine flow on the case is minimized.

The front shell 28 and the rear shell 30 can be assembled by screwing, gluing, clipping, by magnetization, or ultrasonic welding. Of course, other fastening means can be used to assemble the front and rear shells.

For example, the front shell 28 and the rear shell 30 are screwed together. To that end, an internal portion of the front shell has a thread. The thread in the front shell is intended to cooperate with a thread in the rear shell. This allows the case to be easily disassembled to access the test assembly 24 inside the case.

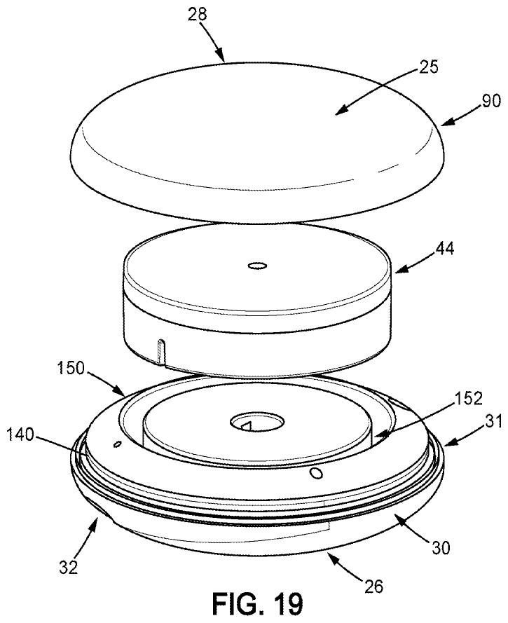

In another example in FIG. 19, an internal portion of the rear shell 30 has a thread 140 intended to cooperate with a thread in the front shell 28. The two shells 28, 30 are assembled by screwing. Alternatively, the assembly of the two shells 28, 30 can be a bayonet system.

A gasket may be present at the joint 31 between the front shell and the rear shell. Thus, the case is waterproof. The inside of the case is thus impervious to urine, water from the water tank 14 or the toilet bowl 16, and any other type of contaminant. Only collection and drain ports connect the outside and inside of the case, as described in more detail below.

Other Functions Supported by the Device

In the example shown in FIG. 5, a removable cover 90 is arranged on the rear shell. The removable cover allows for easy access to the test assembly 24, in particular to a rotatable support 44 of the test assembly. In particular, the removable cover 90 allows the rotatable holder of the test assembly to be recharged.

Here, the removable cover 90 is attached to the rear shell 30 by clipping, screwing or a bayonet mechanism. Of course, other means of attachment may be implemented to secure the removable cover 90 to the rear shell. Alternatively, in another example, the removable cover 90 could be attached to the front shell 28.

The removable cover 90 is arranged in a sealed manner. For example, a joint between the removable cover 90 and the rear shell 30 may include a seal. Thus, the interior of the case 22 remains impervious to urine, water from the water tank 14 or bowl 16, and any other type of contaminant.

In another example, shown in FIG. 18, the removable cover 90 is formed by the front shell 28 of the case 22. The removable cover 90 can then be removed by unscrewing the front shell 28 from the rear shell 30. The case 22 has fewer seams that can be soiled and/or infiltrated by toilet water.

The case 22 has a collection port 32. The collection opening 32 can receive urine flowing by gravity on the outer surface of the case. Urine is collected directly from the faces 25, 26 of the case.

The collection port 32 is located on a lower end 36 of the case 22. The lower end 36 faces the bottom of the toilet bowl 16 when the case 22 is positioned in the toilet bowl 16. This position corresponds to a normal position of use. This position allows urine to be collected by gravity over the majority of the outer surface of the case.

In the present case, a distance D separating the collection port 32 from a lower edge 22a of the case is less than 40 mm, for example less than 20 mm. According to a particular embodiment, the collection hole 32 is arranged a few millimeters above the bottom edge of the case. Alternatively, the collection hole may be on the bottom edge 22a.

The collection port 32 is a circular opening, for example with a diameter of between 0.3 mm and 2 mm. The diameter of the collection port can be chosen to maximize the volume of urine collected from the outer surface of the case.

The case has a drain port 34. The drain port 34 is used to purge the urine analysis device 12 of excess urine.

The drain port 34 may be separate from the collection port 32. To that end, the drain port is also located on the lower end of the case, adjacent to the collection port. The drain port is also a circular opening. The drain port has a diameter between 0.3 mm and 2 mm. In the normal position of use, the drain port is below the collection hole.

The drain port 34 may also be located away from the collection port 32. The position of the drain port 34 may be selected to facilitate access to the drain port by the test assembly 24.

Alternatively, as shown in FIG. 5, the drain port 34 may be the same as the collection port 32. A single port limits the number of openings to the interior of the case. Thus, the risk of introducing contaminants or elements likely to clog the test assembly is reduced.

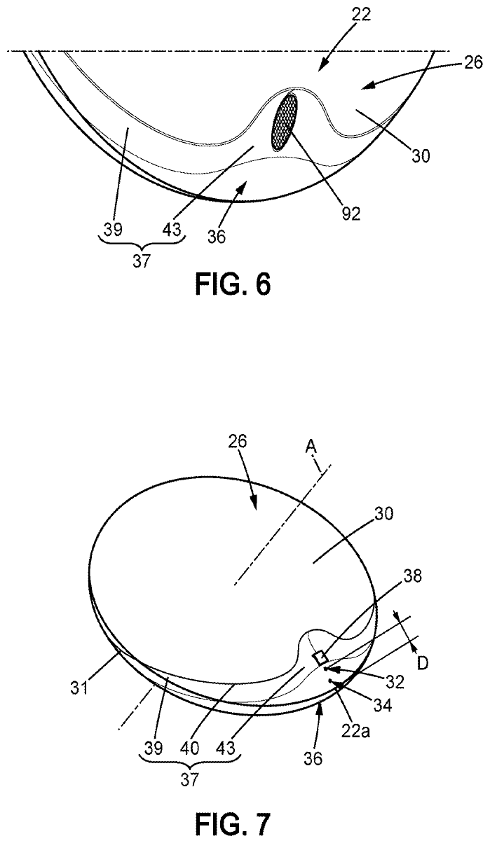

As seen in FIG. 6, the collection port 32 and the drain port 34 may be surmounted by a metal mesh filter 92. The filter covers the ports 32, 34. The mesh filter is for example oblong in shape covering the ports 32, 34. The average mesh opening of the filter is, for example, 20 microns. The filter prevents the introduction of contaminants or elements that may clog the test assembly 24, and filters the urine received in the collection port. Alternatively, the filter could be cleaned by an air flow generated by an air pump.

In the illustrated examples, the collection port and the drain port are located on the rear face of the case. Thus, the collection port and the drain port face the inner wall 16a of the toilet bowl when the urine analysis device is positioned in the toilet. This position allows the collection port and the drain port to be hidden by the front face of the case. Also, this position prevents the introduction of contaminants or elements that could obstruct the test assembly.

The collection port and the drain port are located in a recess 37. The recess 37 has two lateral grooves 39 extending from the seam 31 of the case to a central portion 43 of the recess 37 having the ports 32, 34. The depth of the lateral grooves 39, being the distance from the rear face 26 towards the interior of the case in the direction of the axis A, increases from the joint to the central portion 43. Thus, the recess 37 forms a urine pathway from the front face 25 to the collection port. Advantageously, the indentation allows urine running down the front face to be collected and directed to the collection port. Thus, the volume of urine reaching the collection port from the front of the case is sufficient for the needs of the analysis.

A border 40 delimiting the recess is rounded. In other words, the border is defined by curved surfaces. The edge is free of ridges. Urine in contact with the rear face can flow towards the collection port without falling out of the case or forming air bubbles. This increases the volume of urine reaching the collection port from the back of the case.

The case 22 is not limited to the only embodiments described above with regard to the figures, but is, on the contrary, susceptible of numerous variants accessible to the person skilled in the art.

In particular, the case can have any geometric shape defined by curved lines. In particular, the case can be shaped like a lozenge or an inverted drop. To that end, the case has a point on the lower part to guide the urine towards the collection port.

The collection port and the drain port can be on the front face of the case. In this way, the urine flowing down the front face reaches the collection port more directly.

The collection port and the drain port may be located on a positive relief, such as a projection, or a negative relief, such as a gutter or recess. In general, the relief can be of any geometry that allows urine to be channeled through the case and directed to the collection port without coming off the case or forming air bubbles.

In one example embodiment, the collection port 32 is arranged on the front face 25, while the drain port 34 is located on the rear face 26.

Test Assembly, Injector, Analysis System

Hereinafter, a test assembly 24 utilizing colorimetric strips is described in more detail. The colorimetric strips are herein also referred to as “test strips” 56.

The test assembly 24 is controlled by an electronic control unit 45. The electronic control unit 45 is inside the case 22. The electronic control unit 45 controls the components of the test assembly 24 to perform a urine analysis using the test strips 56 and obtain one or more analysis results.

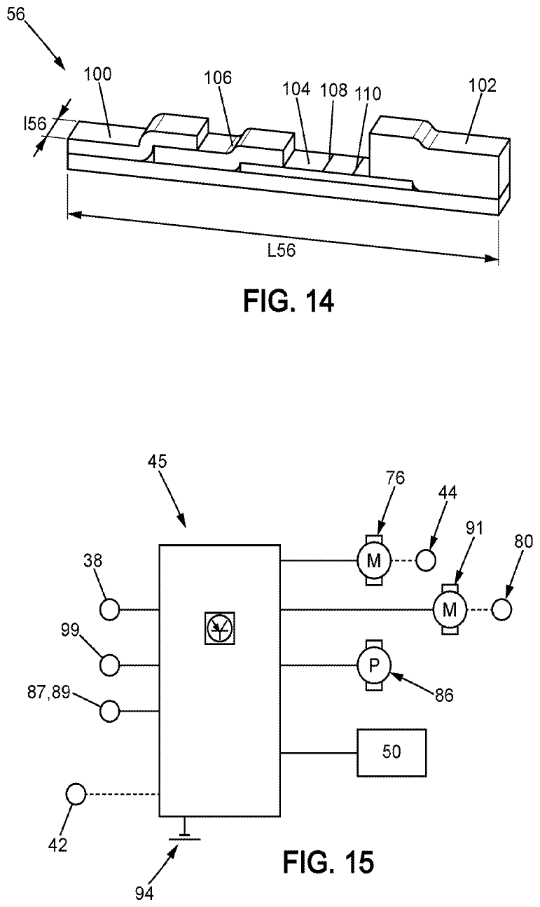

As visible in FIG. 14, the test strips 56 are of the lateral or vertical flow immunoassay type. To that end, the test strips 56 include a sample pad 100 and an absorption pad 102. A nitrocellulose membrane 104 extends between the sample pad 100 and the absorption pad 102. So when a urine sample is introduced onto the sample pad 100, it migrates by capillary action to the absorption pad 102 by passing through a conjugate pad 106, one or more test lines 108, and a control line 110. The conjugate buffer 106, the one or more test lines 108, and the control line 110 contain reagents.

In particular, the conjugate pad 106 includes detection antibodies that are sensitive to compounds in the urine. If the compounds are present when the urine sample passes through the conjugate pad 106, then the antibodies bind to the compounds to form markers. The markers migrate to a test line 108. In particular, the test line includes test antibodies. The test antibodies bind with the markers and retain them on the test line 108. Then, a colored line forms and the density of the line varies depending on the concentration of markers present. The remaining sample migrates to a control line 110. The control line contains control antibodies, allowing to indicate that the sample has passed through the nitrocellulose membrane 104.

For example, the 56 test strips may be ELISA type strips. This type of test strip 56 allows for detection of the pregnancy hormone hCG in urine. Then, the detection antibody may be “mouse monoclonal beta hCG”, the test antibody may be “goat poluclonal anti-mouse IgG” and the control antibody may be “rabbit polyclonal anti-mouse igG”.

Alternatively, the test strips 56 may be conventional colorimetric strips. Then, each test strip includes at least one pad containing one or more reagents sensitive to one or more compounds contained in the urine sample. For example, the compound(s) may be: LH hormone, HCG hormone, leukocytes/nitrites, urobilinogen/bilirubin, protein, pH, specific gravity and/or glucose.

Other types of reactions may use reagents or compounds designed to detect the presence of a particular analyte (e.g., Molecularly imprinted polymers or “MIPs”), including a drug active ingredient or drug active ingredient metabolite in urine. In this case, the device can be used to monitor a user's compliance with a drug treatment, including checking that the user is taking the treatment or alerting the user when the user has failed to take it.

Each test strip 56 is generally rectangular. A width 156 of each test strip 56 may be between 0.5 mm and 3 mm, for example about 1 mm. A length L56 of each test strip may be between 10 mm and 15 mm, for example 12 mm or about 12 mm. Alternatively, each test strip may have any shape, for example square or circular. The shape and dimensions of the test strips allow a large number of test strips to be stored in the urine analysis device 12 (at least 50 strips, or even at least 100 strips). Indeed, it appears possible to store up to 120 test strips, which corresponds to 4 months of analyses when a user performs one analysis per day.

The test assembly 24 operating the test strips specifically comprises one or more rotatable holders 44, an injector 46, a urine delivery means 48, and an analysis system 50.

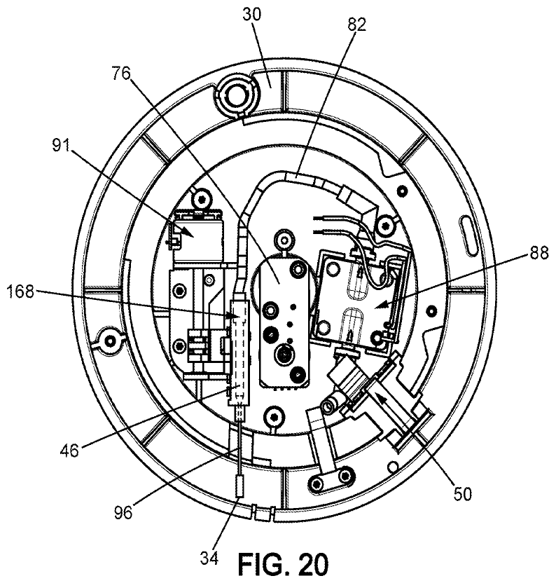

The test assembly 24 is arranged on a base 68. The base allows the injector 46, the rotatable holder(s) 44, the urine delivery means 48 and the urine analysis system 50 to be positioned or even secured in the case. Alternatively, as in the example shown in FIG. 20, the injector 46, the urine delivery means 48 and the analysis system 50 are mounted directly on the case 22, in particular on the rear shell 30 of the case 22. The base 68 is thus formed by the rear shell 30 of the case 28.

The urine analysis device 12 thus comprises a station with one or more rotatable holders 44. The station is thus defined as the urine analysis device 12 excluding the rotatable holder(s) 44. As already explained, the rotatable holder 44 is removable from the station, so that the station and the rotatable holder 44 can be physically separated (e.g., to be manufactured and/or sold independently of each other). An end user or an intermediary can then assemble them. The term cartridge will also be used interchangeably to refer to the rotatable holder.

In the example shown in FIG. 19, a cover 150 covers the components of the test assembly 24, with the exception of the rotatable holder 44. The cover 150 closes the station. The cover 150 comprises a housing 152 for receiving the rotatable holder 44. This configuration protects the components of the test assembly 24 while allowing access to the rotatable holder 44. In this case, the removable cover 90 may be on or formed by the front face of the case to allow access to the housing 152 and change the rotatable holder 44.

Rotatable Holder

The test strips 56 are stored in the rotatable holder 44. In one embodiment, the rotatable holder 44 is of hollow cylindrical shape extending annularly about an axis which is, when the rotatable holder 44 is mounted in the station, the median axis A of the case 22 (for convenience of language, a single axis A will be used to describe the various elements), in practice the rotatable holder is generally rotationally symmetrical about the axis A. The rotatable holder 44 allows a large number of test strips 56 to be stored while being compact enough to be arranged inside the case.

The rotatable holder 44 as shown in the figures extends over a full turn and can make a full turn in the station. However, it may be contemplated, for reasons of space or to allow space to be freed up for other components, that a rotatable holder 44 extends over a portion of a revolution (e.g., less than 180° or 90°) and rotates only a portion of a revolution (e.g., less than 270°). In this case, the number of strips 56 is typically fewer than for the urine analysis device shown in the figures.

The strips are arranged in a circle or portion of a circle, for example at a radial end of the rotatable holder 44 to maximize their number. The positioning in a circle ensures that the strips are all at the same distance from the axis of rotation and, thereby, from the injector 46 or the analysis system 50 (in particular an optical sensor of the analysis system 50, which will be described later). This also ensures that the measurement protocol for each strip is identical. As illustrated in the figures, the strips may be arranged parallel to each other, and more specifically, parallel to the A axis.

Due to its shape and function, the rotatable holder 44 is similar to a barrel.

In the present case, an outer diameter D44 of the rotatable holder 44 may be between 30 mm and 130 mm, preferably about 60 mm. A height H44 of the rotatable support, measured in the direction of the axis A may be between 12 mm and 40 mm, preferably about 14 mm. A ratio between the diameter D44 of the rotatable support 44 and the diameter D22 of the case 22 may be greater than or equal to 0.3, preferably greater than or equal to 0.5. This provides a very compact solution with respect to the large number of test strips available.

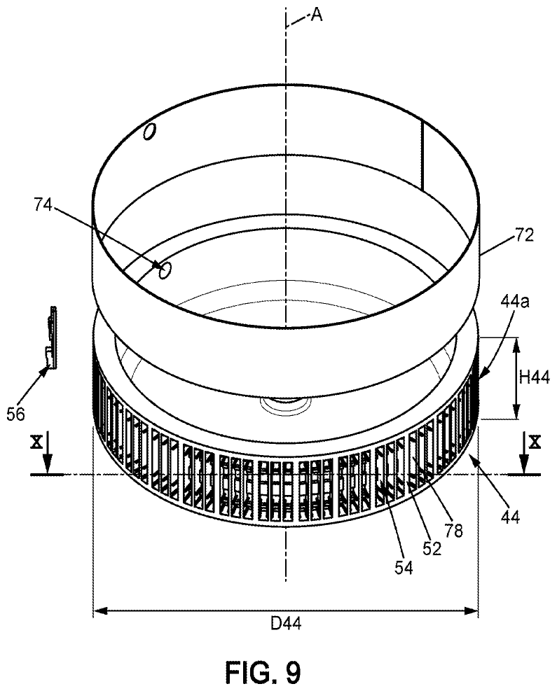

In a first example, the test strips 56 are received in an outer wall 44a of the rotatable holder 44 (illustrated in particular in FIGS. 9-13). Accordingly, the number of test strips 56 that can be stored by the rotatable holder is further increased.

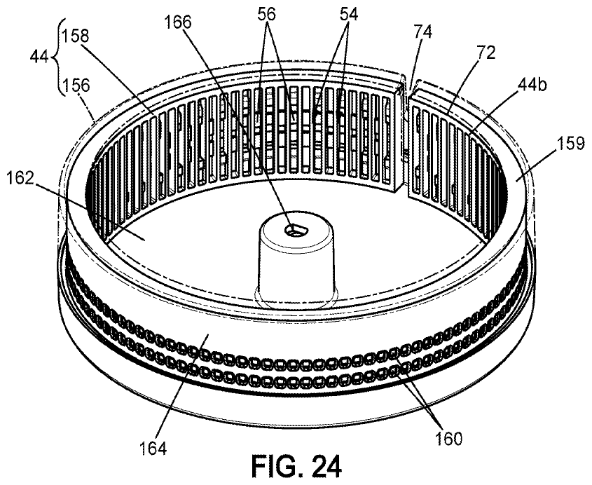

Alternatively, in an example described in more detail below (with reference to FIGS. 24 through 28), the test strips may be stored in an inner wall of the rotatable holder. This arrangement prevents the user from touching the strips when manipulating the rotatable holder. This configuration also allows for flexibility in positioning the injector and the analysis system relative to the rotatable holder.

According to another example not shown, the rotatable holder 44 could be a washer, with an axis coinciding with the median axis A of the case. The washer then extends radially, in a plane substantially normal to the median axis A of the case. The test strips can then be stored on one face of the washer, normal to the axis A. This configuration makes it possible to adapt the rotatable holder to different case shapes. Therefore, the rotatable holder can be implemented in various urine analyzers.

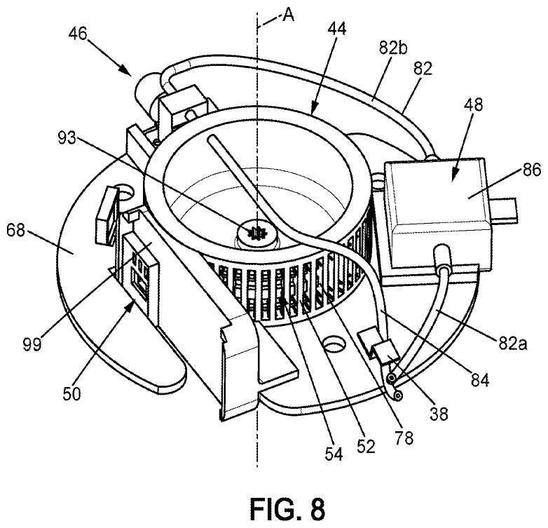

As illustrated (particularly in FIGS. 8 to 10), the outer wall 44a of the rotatable holder has a succession of grooves 52 bounded by small walls 78. The small walls form housing 54 for the test strips. The small walls allow a housing 54 to be isolated from adjacent housings along the circumferential direction. Thus, the housings 54 can separate the test strips 56 to be used in successive analyses. Used test strips are separated from new test strips.

Here, the grooves 52 extend in the direction of the A axis, substantially over the entire height of the rotatable holder 44. Thus, the rotatable holder may have between 40 and 150 housing, preferably between 60 and 120. Thus, a large number of test strips can be stored in the rotatable holder.

Alternatively, the grooves 52 could extend in a radial direction. This configuration appears interesting to further increase the number of housings of the rotatable holder 44, and store more test strips.

Each housing 54 may receive a single test strip 56. All of the test strips in the housings may be of the same type. The same type means that they are sensitive to the same compounds in the urine. The rotatable holder is then adapted for a specific analysis.

Alternatively, the test strip 56 received in housing 54 may be of a different type than the test strip received in the adjacent housing. Thus, multiple types of analyses, requiring different types of test strips, can be performed from the same rotatable holder 44.

Alternatively, each housing 54 may contain a plurality of test strips of different types. Thus, multiple types of tests can be performed from a single housing.

The rotatable holder 44 includes an opening 74. The opening allows a needle 96 of the injector 46 to pass through the rotatable holder, in particular to perform an evacuation of the urine contained in the urine analysis device 12.

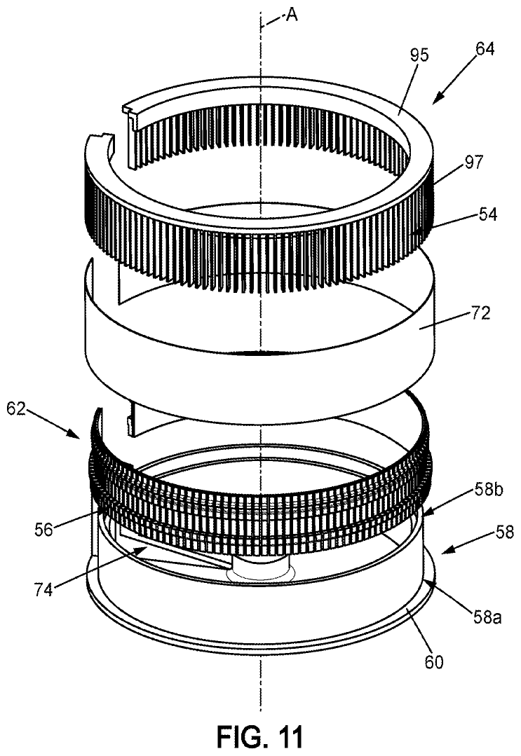

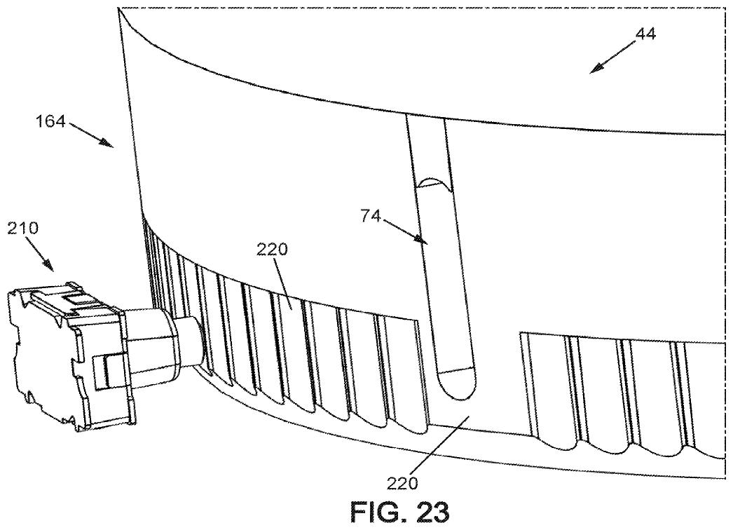

In the example shown in FIG. 9, the opening 74 is a circular opening, extending radially across the rotatable holder. Alternatively, the opening 74 may be a slot extending the full height H44 of the rotatable holder. Alternatively, the opening may be oblong, as shown in FIG. 23. Alternatively, the opening 74 may be defined by an angular sector, as illustrated in FIG. 11, 24, or 28. The angular sector may be in the form of a notch formed in the rotatable holder (see FIG. 24 for example). The shape of the opening allows for easier fabrication of the rotatable holder, particularly during injection molding fabrication.

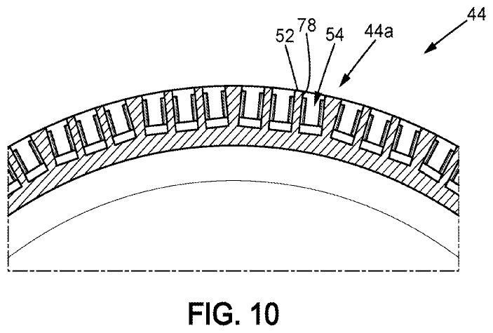

In the example shown in FIG. 9, the rotatable holder 44 is made of a single piece. As is more apparent in FIG. 10, the grooves 52 are then arranged in groups of 3 in a plane tangential to the outer wall 44a of the rotatable holder. This arrangement limits the number of inserts required to manufacture the rotatable holder during an injection molding process.

Alternatively, in the example shown in FIG. 11, the rotatable holder 44 is formed by two parts. A first part 58, called the ‘armature’, takes the form of a hollow cylinder or ring extending around the axis A, being coaxial with it. An annular rib 60, extending radially outward, surrounds one end 58a of the first armature part 58. Thus, the test strips 56 can be positioned on the first armature part 58. The test strips may then be joined to form a strip 62. A second part 64 includes a ring 95. The ring 95 is intended to cooperate with the end 58b of the first armature part 58, opposite the first end 58a in the direction of the axis A, to form the rotatable holder.

The ring 95 has a succession of projections 97 extending in the direction of the A-axis, in the assembled position, to the annular rib 60 of the first armature part 58. The projections 97 separate the test strips 56 of the strip 62. Thus, the projections 97 form the small walls of the housings 54 receiving the test strips. This configuration facilitates handling and assembly of the test strips in the rotatable holder.

Lid

Each housing 54 is covered and closed by a lid 72. The lid seals the test strips received in a housing from the outside environment and from adjacent housings. Thus, before an analysis, the reagents in the test strips are protected from a possible contamination. In addition, after an analysis, the lid 72 may contain urine introduced into the housing 54.

As particularly visible in FIGS. 9 and 11, the lid 72 may take the form of a continuous film. The film is adhered to the outer wall 44a of the rotatable holder to cover the housing 54. This configuration facilitates the installation of the lid 72 on the rotatable holder.

Such a construction allows a simple and automatable assembly from relatively simple components. The cost of a rotatable holder equipped with test strips is thus low.

Alternatively, each housing 54 may be covered by a separate lid 72. This configuration limits the risk of contamination of test strips received in two adjacent housings.

Alternatively, the test strips 56 may be individually encapsulated. This configuration appears particularly attractive when the test strips are joined to form a strip 62. This is because the strip 62 can be assembled in the rotatable holder without the need for an additional lid 72. This facilitates the assembly of the urine analysis device 12.

Here, the lid 72 is made of an inert material. For example, the lid may be made of silicone or acrylic. Preferably, the lid is of medical grade, to avoid contamination of the test strips with undesirable products contained in the lid. Thus, the reagents in the test strips are kept intact before analysis.

In addition, the lid 72 is transparent, with a transparency rate preferably greater than 99%. Then, a colorimetric analysis can be performed on a test strip through the cover.

Motor

The rotatable holder 44 is assembled on the shaft of a motor 76 (FIG. 4) to be rotated about the A axis. The rotatable holder can then be selectively positioned to align a test strip in front of the injector 46 or in front of the analysis system 50. Thus, the use of the rotatable holder provides a simple moving element with a single axis of rotation. In addition, this configuration reduces the constraints related to the positioning and arrangement of the injector 46 and the analysis system 50 in the analysis device 12.

Alternatively, the motor 76 could be non-aligned to the A-axis of the rotatable holder hub 44. As shown in FIG. 20, for example, the motor 76 may be offset from the A-axis, with a gear reducer connecting the rotor of the motor 76 to the rotatable holder 44. The gearing may allow for greater accuracy in the angular position of the rotatable holder 44.

It is not impossible to have the motor 76 and its output shaft arranged radially outside the rotatable holder 44. For example, the outer wall of the rotatable holder 44 may have a succession of teeth cooperating with teeth connected to the motor shaft 76. Alternatively, the rotatable holder 44 could be attached to a disk having the succession of teeth.

The motor 76 can drive the rotatable holder 44 in a clockwise or counterclockwise direction. The rotatable holder can then quickly reach the desired position, following the shortest trajectory. This further reduces the constraints associated with the positioning and arrangement of the injector 46 and the analysis system 50 in the analysis device 12.

The motor 76 is for example a stepper motor. Thus, the stepper motor provides controlled indexing of the rotatable holder. Alternatively, the motor can be a DC motor.

### Markers

The angular position of the rotatable holder 44 may be controlled by detecting one or more markers on the rotatable holder. The marker(s) allow for precise control of the position of the rotatable member in the urine analysis device. The electronic control unit 45 is coupled to a sensor configured to detect the presence in a particular position of the one or more markers.

The rotatable holder can be inserted into the urine analysis device in a random manner, and the positioning of the rotatable holder can advantageously be carried out automatically, after an initialization step consisting of a “blind” rotation until at least one of said markers is located.

A point marker can be provided on the rotatable holder, this point marker acting as a ‘zero’ angular reference. From the knowledge of this angular reference position called ‘zero’. Then the stepper motor control memorizes the number of steps taken in each direction, allowing the electronic control unit to continuously follow the current angular position of the rotatable member. This process is carried out in an open loop, but a possible readjustment can be provided each time the ‘zero’ marker is in front of the sensor.

In one example, the marker(s) may be magnetic markers, for example.

In another example, the marker(s) may be optical markers, for example. The optical markers may be lines or bar codes on the rotatable holder. The optical markers are intended to cooperate with an optical sensor 99. Advantageously, the use of an optical sensor can also identify the type of test strips in the rotatable holder. In addition, the same optical sensor 99 can perform colorimetric analysis on the test strips. This configuration reduces the complexity and cost of manufacturing the urine analysis device by limiting the number of components used in its operation.

Cam Locking and/or Counting Mechanism

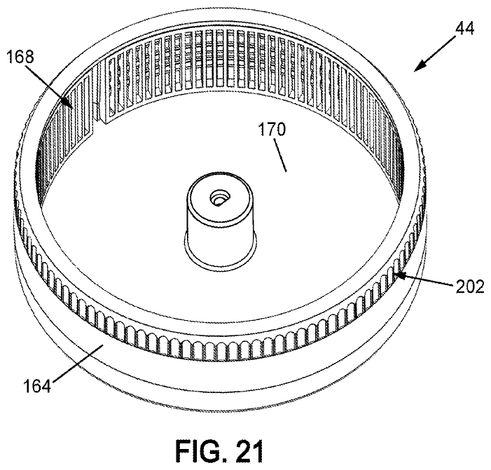

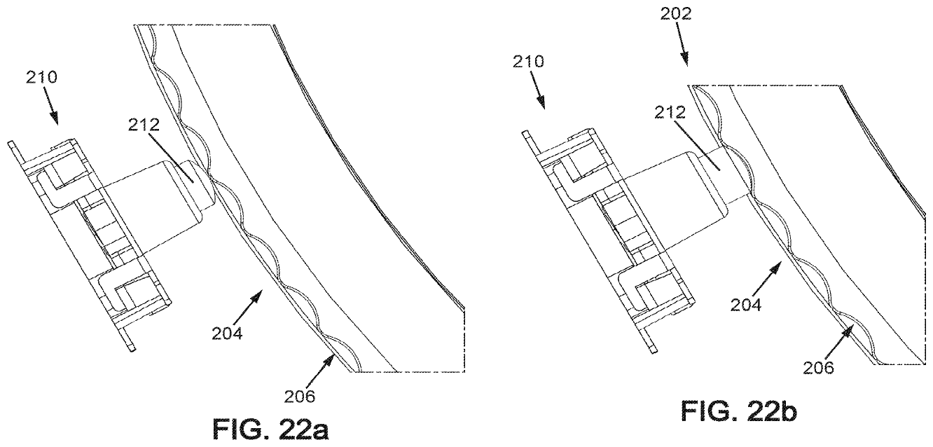

In an embodiment illustrated in FIGS. 21-23, a blocking and/or counting mechanism is incorporated into the urine analysis device 12. The rotatable holder 44 may include, on an outer periphery (typically an outer periphery of an annular portion 162 or a cylindrical portion 164, which will be described later), a cam 202 having alternating highs 204 and lows 206. In other words, the radius of the rotatable holder 44 varies angularly at the cam 202. Opposite this cam 202 is a follower 210 (the follower is part of the station) configured to identify the highs 204 and lows 206 of the cam 202. The follower may typically include a translating rod 212 that is pushed in the direction of the cam 202 by a spring (not visible in the figures) by default, so that the rod 212 moves in accordance with the highs 204 and lows 206 of the cam 202, as illustrated in FIGS. 22a, 22b, respectively. In one example, the follower has a binary output, corresponding to a high or a low. Thus, it is possible to count the number of highs 204 and lows 206 passed as the rotatable holder 44 rotates in the station. Each low 206 (alternatively each high) is associated with (e.g., radially aligned with) a test strip 56 on the rotatable holder 44 (or a housing 54 receiving the strip), so that the counting mechanism can tell exactly how many strips have passed the follower 210 and thus, using a marker (either zero or the last known position), can tell which strip is at the injector 46 and/or the optical sensor 99.

The cam 202 is thus an embodiment of the aforementioned markers.

The follower 210 may be a microswitch, such as a microswitch with a binary output.

In particular, such a counting mechanism eliminates the need for a stepper motor and facilitates the use of a conventional, DC type motor. In addition, the motor 76 can be controlled using data obtained by the follower 210, which ensures that any motor-related inaccuracies will not upset the urine analysis device 12.

In one embodiment, the cam may have a one-way pattern, so that the cam and follower function as a pawl allowing rotation in only one direction.

As shown in FIG. 23, at the opening 74, the cam 202 may have a portion 220 that is flat or has a different pattern than the highs and lows of the rest of the cam 202, so that the follower 210 can identify the angular ‘zero’ mark. For example, fora given motor 76 rotational speed, the follower 210 will maintain the same output for a period of time that is greater than a threshold or greater than the time it takes to pass a high or low.

In this embodiment, the optical sensor 99 is not used to count or identify the angular position of the rotatable holder 44. To simplify the implementation of the cam 202, the follower 210 may be angularly positioned at the same location as the optical sensor 99, i.e., when the follower is facing a low (or high) of the cam, the optical sensor 99 is also facing the strip and housing that are associated with that low (or high) of the cam.

The cam 220 and follower 210 also allow, in addition to or alternatively to the counting mechanism, to generate a stop that opposes the rotation of the rotatable holder 44. In particular, when the direction of rotation of the motor 76 is reversed, the gear chain may experience a backlash that impairs the accuracy of the system. By blocking the rotation of the rotatable holder 44, it is possible to ensure that the backlash is compensated for before any movement of the rotatable holder 44. In addition, because the motor 76 can be controlled using data from the follower 210, the motor 76 can slip, slow down, speed up, or skip one or more gear teeth without generating accidental shifts between the test strips 56. The blocking mechanism may also be activated during an injection of urine onto the test strip 56, or even during the strip analysis phase. To increase the blocking force, the follower 210 may then include a blocked mode, which prevents translation of the rod 212 and thereby helps to immobilize the rotatable holder 44. In this embodiment, a radial alignment of a stocking with a strip is preferred.

Alternatively, the cam 202 and follower 210 can be reversed (follower mounted on the rotatable holder and cam mounted on the station). However, this solution is less convenient to implement and less economical.

Other types of followers can be mounted in the urine analysis device (with a wheel that rolls on the cam, with a flexing blade, etc.). Similarly, instead of a binary microswitch, the follower can have more outputs.

Mounting of the Rotatable Holder

The rotatable holder 44 is removably mounted in the urine analysis device. Thus, the rotatable holder can be removed from the case. The rotatable holder can be replaced, in particular to refill the test assembly 24 with test strips or to change the type of test strips. So, the urine analysis device is modular and versatile.

In the example shown in FIG. 8, the rotatable holder 44 has a female spline sleeve 93 adapted to be assembled to a male sprocket on the motor shaft 76. The rotatable holder can then be easily removed and inserted from the case by translation along the A axis, without compromising the rotation of the rotatable holder.

As illustrated in FIG. 5, the case 22, including a removable cover 90, is particularly suitable for removing the rotatable holder 44 from the case. Indeed, the rotatable holder is easily accessible. In addition, the removable cover protects the remainder of the test assembly 24 when the rotatable holder is removed.

As described above, in one example, the removable cover 90 is arranged on the front face 25 of the case. In another example, the removable cover 90 is arranged on the rear face 26 of the case. In yet another example, the removable cover 90 is formed by the front face 25 of the case.

As shown in FIG. 5, a groove 90a is provided on the outer face of the removable cover. This groove 90a allows the removable cover 90 to be rotated by inserting a coin, for example. The depth of the groove can be from 1 mm to 1.5 mm. Its length is close to the diameter D44 and its width can be from 2 mm to 2.5 mm.

Alternatively, as shown in FIG. 19, the removable cover 90 is removed by unscrewing the front shell 28 from the rear shell 30.

Double Barrel Variant

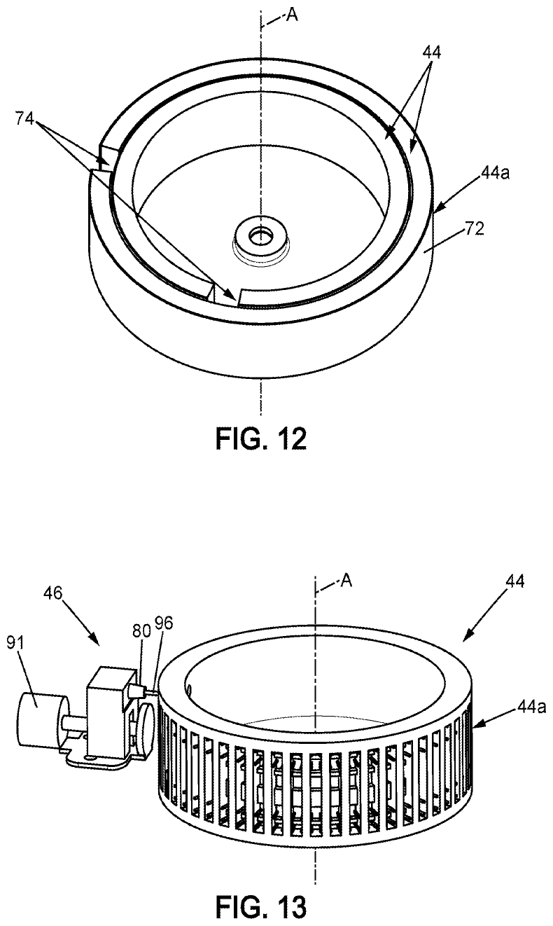

In the example shown in FIG. 12, the test assembly 24 includes two rotatable holders as described above, namely a first rotatable holder 44 on the outside and a second 442 arranged inside the first. The two rotatable holders 44 are coaxial along the A axis. Two rotatable holders allow more test strips to be stored in the case. So, each rotatable holder 44,442 has an opening 74 in the form of a slot. The slots further allow the needle 96 to pass through to access the housings 54 of either of the rotatable holders 44,442.

Each rotatable holder 44,442 is controlled in angular position by its own motor, two motors being arranged in coaxial configuration.

Rotatable Holder Variant

FIGS. 24 through 28 illustrate another example of a rotatable support 44. Here, the strips 56 are stored in an inner wall 44b of the rotatable holder 44. The rotatable holder 44 may be received in the housing 152 of the cover 150 covering the other components of the test assembly 24.

The rotatable holder 44 comprises a receptacle 156 and a separator 158.

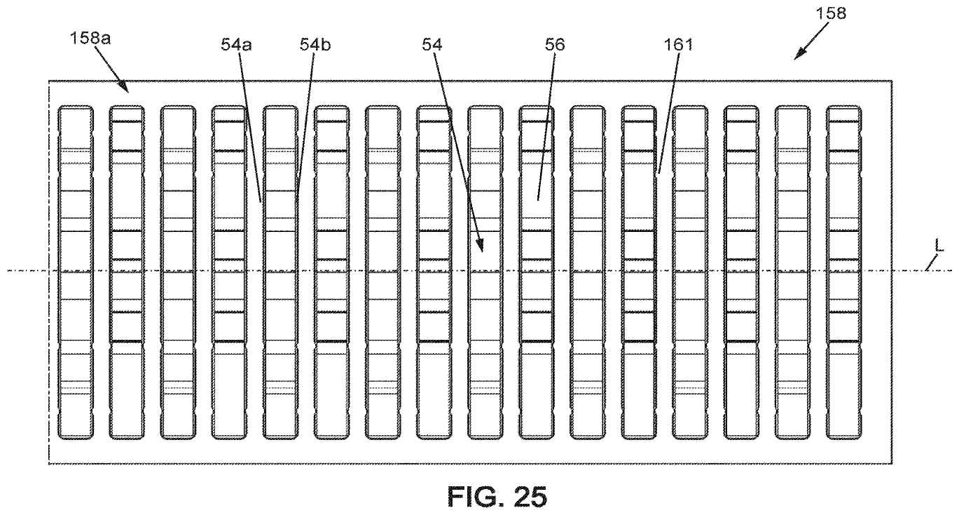

The separator 158 is a flexible part, in particular made of elastomer in the form of a strip or band intended to be wound into the receptacle 156. As illustrated in FIGS. 25, 26 and 27, the band (and also the separator 158) extends along a longitudinal direction L. The separator 158 comprises a first face 158a comprising the housings 54 receiving the test strips 56. The first face may be covered by the lid 72 (schematically visible in FIG. 24). The housings 54 are sealed. The test strips 56 can then be inserted into the housings 54 on a flat surface. Assembly is simplified.

Each housing 54 may include two parallel side walls 54a, 54b (i.e., the walls perpendicular to the longitudinal direction L), to facilitate insertion of the strips 56. In addition, once the separator 158 is rolled up and mounted in the receptacle 156, the circular curvature tilts the side walls 58a, 58b which gradually close toward the surface of the separator (i.e., toward the lid 72). This allows the strips 56 to be trapped within their housing 54 in an efficient and simple manner. Alternatively or complementarily, each housing may include, on at least one side wall 54a, 54b, a rib 161 (typically integral with the separator 158) that requires the strip 56 to be slightly constrained in order to insert it into its housing 54. This rib 161 helps to keep the strip inside its housing. In FIG. 25, each housing 54 comprises four pairs of ribs 161, each pair comprising two ribs facing each other.

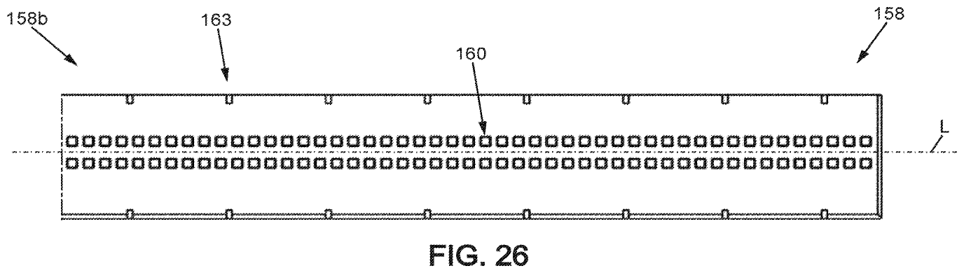

As illustrated in FIG. 26, the separator 158 also comprises a second face 158b, opposite the first face 158a. The second side 158b has through holes 160. The holes open into the housing 54 receiving the test strips 56. In particular, each housing 54 comprises at least one hole 160. The holes 160 are aligned with areas of interest on each test strip 56, for example, the test lines 108. The holes 160 allow light to be guided to the strips 56 for colorimetric analysis. This allows the strip to be positioned between the light source 176, 178 on one side and the optical sensor 99 on the other side. Light can be guided through the holes 160 to the areas of interest on the test strip 56. Thus, two holes 160 may be provided per housing 54, to analyze two areas of interest on a test strip 56.

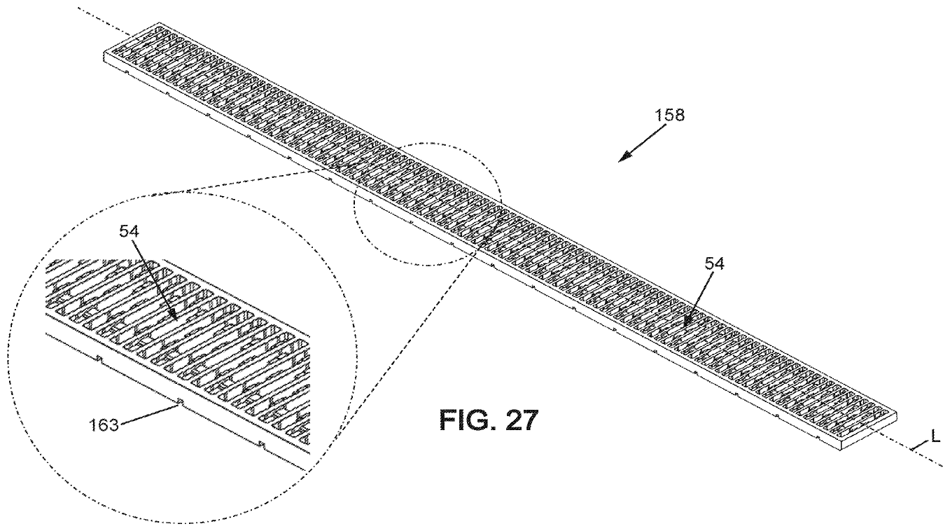

The separator 158 may include, for example on the second side 158b, at least one recess 163 or protrusion (recess visible in FIGS. 26 and 27). Complementarily, the receptacle 156 comprises a projection or recess (respectively) to cooperate with the separator recess or projection (respectively). In particular, this ensures that the housings 54 (and thus the strips 56) are in radial alignment with the lows (or highs) of the cam 202. A plurality of recesses/protrusions 163 may be provided between the separator 158 and the receptacle 156 to ensure that each housing 54 is properly radially aligned with a low or high of the cam 206. Indeed, the separator 158, being made of a flexible material, may exhibit manufacturing irregularities, which the recess/profile pair(s) allow to compensate for, by keeping stretched or compressed certain portions of the separator 158 once the latter is in place.

In addition to or as an alternative to the recess/protrusion 163, the separator 158 may be bonded to the receptacle 156. An adhesive that is transparent to the frequencies used for optical analysis is typically used.

As illustrated in FIG. 24, the receptacle 156 comprises an annular portion 162 and a cylindrical portion 164, radially external to the annular portion 162

The cylindrical portion 164 is transparent, or comprises transparent areas. The cylindrical portion 164 contacts the separator 158, in particular the second side of the separator 158. In a colorimetric analysis, light can pass through the cylindrical portion 164 to analyze the test strips 56.

In particular, the cylindrical portion 164 may be made of polycarbonate. Indeed, polycarbonate has good light transmission properties, while remaining relatively inexpensive and compatible with an injection molding process.

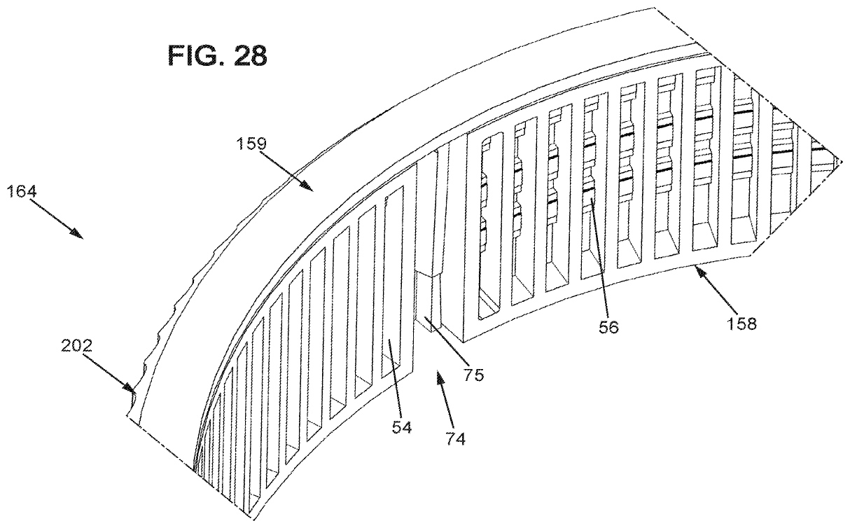

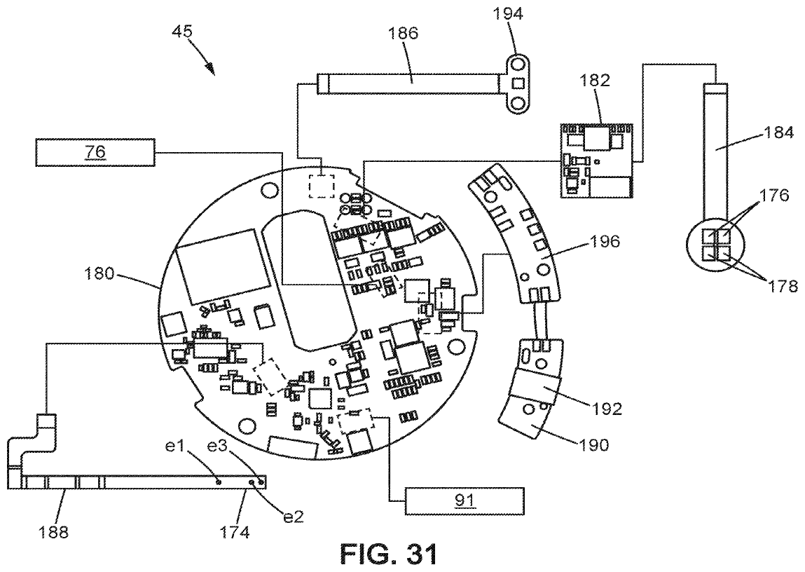

The annular portion 162 of the receptacle 156 forms a base for carrying the separator 158. The cylindrical portion 164 receives the separator 158. The separator 158 is locked in translation along direction A by contact with the annular portion 162. On the other side, as shown in FIG. 31, an annular flange 159 extending from one end of the cylindrical portion 164 and radially inwardly blocks translation along the other direction A. The annular flange 159 extends, for example, a distance similar to the thickness at the separator 158. To block rotation of the separator 158 in the receptacle 156, in addition to the previously described adhesive and the previously described recesses/protrusions 163 and/or as alternative to these, at least one stop 75 (see FIG. 28) may be mounted on the receptacle 156 (typically on the cylindrical portion 164). For example, the stop 75 is located on the cylindrical portion 164 at the opening 74 so that the end of the separator 158 contacts the stop 75. Another stop may be provided symmetrically on the other side of the opening, against which the other end of the separator may abut.

The annular portion 162 further comprises a female sleeve 166 for attaching the motor 76. The female sleeve 166 forms the hub for receiving the shaft of the motor 76 or an interposed gearbox at its center. Here, the female sleeve 166 is aligned with the axis A of the rotatable holder 44. However, in the example where the motor 76 is radially external to the rotatable holder 44, the annular portion 162 could be devoid of the female sleeve 166. The annular portion 162 could be mounted by any type of pivotal connection relative to the case 22. The annular portion 162, when not provided with a through hole at the axis A, may be similar to a disk.

Alternatively, the optical markers here may be the opening 74 of the rotatable holder 44 and each of the holes 160 of the separator 158. Indeed, the opening 74 of the rotatable holder 44 may act as a ‘zero’ angular marker and each hole 160 may then provide the angular position of the rotatable holder 44 relative to the ‘zero’ angular marker.

### Infector

The injector 46 includes an automated syringe 80, on which the needle 96 is oriented toward the housing(s) of the rotatable holder(s). The injector 46 is designed to pierce the lid 72 and inject a urine sample onto a test strip.

In the example shown in FIG. 13, the injector 46 is arranged outside of the rotatable holder. The automated syringe 80 extends radially in the direction of axis A. Thus, the needle 96 points towards the outer wall 44a of the rotatable holder. Alternatively, for example when the test strips are stored in an inner wall of the rotatable holder, the injector could be arranged radially inside the rotatable holder.

The automated syringe 80 injects, through the needle 96, a controlled volume of urine onto a test strip, for example between 2.5 microliters and 3.5 microliters. Thus, the automated syringe 80 injects a sufficient volume of urine onto a test strip to perform a conclusive analysis without risking an overflow of urine from the housing.

The automated syringe 80 can inject a controlled volume of urine onto the test strip 56 in two stages. For example, the automated syringe 80 can inject between 2.5 microliters and 3.5 microliters twice. This allows for a reaction and migration time of the urine on the test strip 56.

The needle 96 has a side opening. The side opening allows the passage of the urine sample from the syringe to the housing. The side opening prevents the needle tip from becoming blocked during multiple successive piercings.

The injector 46 may include a first sensor 89. The first sensor is arranged upstream of the automated syringe 80. The first sensor can then detect whether urine is being received in the automated syringe. The injector may further include a second sensor 87. The second sensor 87 is arranged in the automated syringe 80, proximate to the needle 96. Then, the second sensor can verify that the automated syringe contains urine to perform an injection. The sensor(s) 89, 87 can then monitor the injection of urine onto the test strip. Alternatively, the injector 46 may include a urine volume monitoring means 168, as will be described in more detail below.

The automated syringe 80 is arranged on a linear motor 91. The linear motor allows the automated syringe to be moved radially. At rest, the automated syringe is moved away from the rotatable holder to allow rotation of the rotatable holder. Additionally, the automated syringe may be moved closer to the rotatable holder to reach a test strip. The automated syringe 80 may also pass through the opening 74 of the rotatable holder, particularly to drain excess urine from the automated syringe.

In addition, in the example having two rotatable holders 44,442 the automated syringe 80 can be moved radially to reach a test strip on either rotatable holder 44. Thus, an overtravel is provided to reach the second rotatable holder 442.

Pump and Piping

According to a first example, the urine delivery means 48 includes a collection channel 82, a purge channel 84 and a pump 86. The collection channel connects the collection port 32 to the injector 46, in particular to the automated syringe 80. Thus, urine can reach the automated syringe from the collection port. The purge channel 84 connects the injector, in particular the automated syringe 80, to the drain port 34. The purge channel then allows the urine in the urine analysis device to drain.

Here, the purge channel 84 is arranged radially inwardly of the rotatable holder. The automated syringe 80 accesses the purge channel 84 through the opening 74 of the or each rotatable holder. Urine can then be evacuated by injection of the automated syringe into the purge channel. The urine analysis device may be without components particularly dedicated to urine evacuation. The complexity of the urine analysis device is reduced.

Here, the automated syringe 80 is inserted into the purge channel 84. Thus, the connection between the automated syringe and the purge channel is tight. The risk of leakage when urine passes from the automated syringe to the purge channel is reduced.

Preferably, the purge channel 84 is hydrophobic, allowing for better evacuation of the urine. This reduces the risk of contamination of the urine transported between two successive urine collections.

The pump 86 is arranged between a first portion 82a and a second portion 82b of the collection channel 82.

The pump 86 can draw urine from the collection port 32. For example, the pump draws between 5 microliters and 1 mL, preferably about 20 microliters. In addition, the pump can deliver a sufficient volume of urine to the injector 46 to be able to perform a conclusive analysis. A suction rate of the pump 86 is selected based on the diameter of the collection port. Advantageously, the pump can draw urine from the collection port to the injector without forming air bubbles.

In another phase, after urination and outside of the flushing sequence, the pump 86 may also draw air from the collection port 32. The air then passes through the urine delivery means 48 and the automated syringe 80 to the drain port 34. The pump then expels urine or water from the urine analysis device. Urine collected for analysis is then protected from possible contamination by toilet water or from a previous collection.

According to a further or alternative embodiment, it may be provided that the pump 86 may draw water upon activation of the toilet flush to discharge urine contained in the urine analysis device.

The pump 86 may be of various possible types. The pump 86 may be a miniaturized peristaltic pump. The pump 86 may be a miniaturized pneumatic pump system as detailed below.

In the case where the pump 86 is a miniaturized pneumatic pump, this pneumatic system is configured to create a vacuum to draw urine from the collection port 32 and then a positive pressure to push the urine to the injector 46 and the purge channel. In addition, this pneumatic system comprises an internal buffer space interposed between the first portion 82a and the second portion 82b of the collection channel 82, a supply valve (check valve) and a discharge valve (check valve).

When this pneumatic system creates a vacuum, it draws fluid from the collection port 32 via the first portion 82a and the inlet valve; the fluid drawn may then be urine and/or water and/or air.

The fluid thus drawn in is stored in the internal buffer space of the pneumatic system. Then, this pneumatic system creates an overpressure, and this pushes fluid, from the internal buffer space, through the discharge valve and the second portion 82b, towards the injector 46.

Thanks to the non-return valves, the pneumatic system can be without controlled valves, which reduces the complexity of the urine analysis device.

The pump of the pneumatic system can be a rotary type pump here, with the direction of rotation respectively and selectively providing vacuum or overpressure. The pump of the pneumatic system can also be a piezoelectric type pump.

When urine is aspirated, the injector may be set to the purge position to release air until urine reaches the needle threshold.

The presented solution allows for precise control of the volume delivered to the urine analysis device.

Pump and Pipe Variant

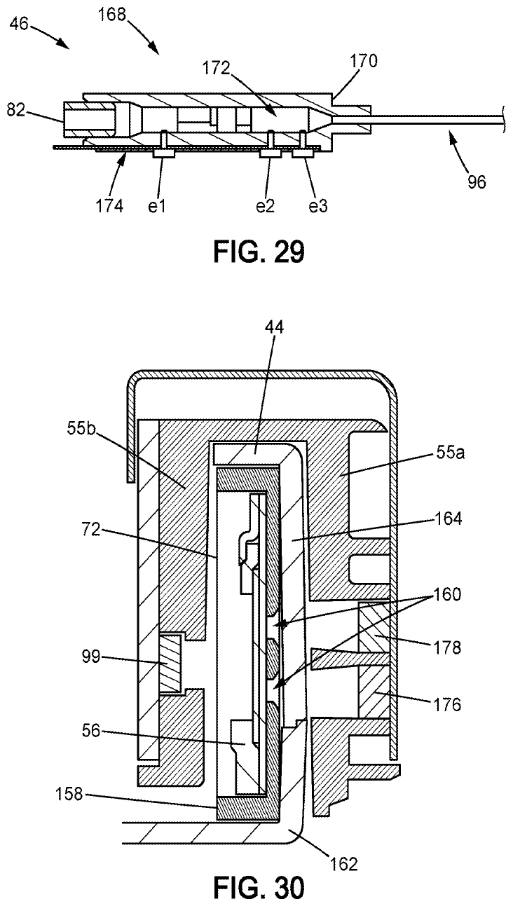

A second example of a urine delivery means 48 is described next with reference to FIG. 29.

In this example, the injector 46 is radially internal to the rotatable holder 44. The direction of translation of the injector syringe may be at an angle (e.g., between 5° and 45° or between 5° and 30°) to a radial direction that passes through the end of the syringe in the retracted position) for space reasons. The injector 46 may directly access the drain port 34 through the opening 74 of the rotatable holder 44. The urine delivery means may be devoid of the purge channel 84. Urine can be discharged by injection through the drain port 34.

Furthermore, in this example, the pump 86 is also radially internal to the rotatable holder. The arrangement of the pump 86 and injector 46 frees up space external to the rotatable holder 44, so that the diameter D44 of the rotatable holder 44 can be increased. An increased number of test strips 56 can be stored by the rotatable holder 44.

Urine Control Device

As visible in FIG. 29, the injector 46 may include a urine monitoring means 168. The urine monitoring means 168 is intended to provide a measurement of volume and flow rate of urine flowing through the needle 96.

The urine monitoring means 168 comprises a sleeve 170. The sleeve 170 is arranged between the needle 96 and the urine collection channel 82. The sleeve 170 is a hollow cylindrical part. The sleeve 170 defines an internal cavity 172 through which urine can flow to the needle 96.

A flexible printed circuit 174 is mounted on an outer wall of the sleeve 170. Three conductive electrodes e1, e2, e3 extend from the printed circuit 174 into the cavity 172. The electrodes can detect urine flowing into the cavity 172 by generating an electrical signal.

A reference volume is defined by the distance between a first electrode e1, in the vicinity of the needle 96, and a second electrode e2, remote from the needle 96, and the cross-sectional area of the cavity 172. The reference volume is, for example, 20 μL. The pump 86 can be activated to deliver urine to the needle 96 until urine is in contact with both the first and second electrodes e1, e2. When urine is in contact with both the first and second electrodes e1, e2, an electrical signal can flow in a closed circuit between the first and second electrodes e1, e2. The closed circuit indicates that the reference volume is reached in the cavity.

Note that the reference volume is greater than the controlled volume of urine intended to be injected onto a test strip 56.

The reference volume comprises a pre-injection volume. The pre-injection volume may be circulated through the needle 96 to the drain port 34. The pre-injection volume ensures that the needle 96 is loaded with urine prior to an analysis. This ensures that the urine in the needle 96 is free of air bubbles that could reduce the volume of urine actually injected onto the test strip 56.

The reference volume also comprises a safety volume to take into account the electromagnetic tolerances of electrodes e1, e2, e3.

In addition, when the pump 86 is activated, the time elapsed between a contact of the urine with a third electrode e3, disposed between an end of the sleeve 170 connected to the needle 96 and the first electrode e1, provides a measure of urine flow rate. The urine flow rate can determine the activation time of the pump 86 required to inject the controlled volume of urine onto the test strip 56. Indeed, the relationship between urine flow rate and pump 86 activation time is linear. Determining the activation time eliminates the need to calibrate the pump 86.

Other Functions Supported by the Device

In one embodiment, the test assembly 24 includes a urine presence sensor 38. The urine presence sensor is arranged in the vicinity of the collection port. The urine presence sensor then detects when urine is present in the vicinity of the collection port.

According to one embodiment, the urine presence sensor 38 could form a ring around the collection port. The integration of the urine presence sensor into the urine analysis device is then discrete.

The urine presence sensor 38 can be a temperature sensor, for example a thermistor. The temperature sensor can indeed distinguish between urine and water from the toilet. In addition, the temperature sensor may also be operated to measure the temperature of the urine. The temperature of the urine can be used to detect periods of fertility, for example, by comparison with one or more reference curves. The use of a temperature sensor reduces the number of components used by the test assembly to perform an analysis. The complexity and cost of manufacturing the urine analysis device is reduced.

Alternatively, the urine presence sensor 38 may be any type of liquid sensor, such as a capacitive or resistive type sensor. Then, a temperature sensor is separate from the urine presence sensor. The temperature sensor may be dedicated to measuring the temperature of the urine, in particular to detect a fertile period.

Analysis System

The analysis system 50 performs colorimetric analysis on the test strips. By “colorimetric analysis” is meant a measurement of absorbance or fluorescence under a predetermined illumination, either in transmission or reflection. The analysis system can then determine one or more analysis results.

Here, the analysis system 50 is arranged radially outside of the rotatable holder. Alternatively, for example when the test strips are stored on an inner side of the rotatable holder, the analysis system could be arranged radially inside the rotatable holder. In one particular example described later, the analysis system 50 overlaps the rotatable holder 44.

Alternatively, the analysis system 50 may be arranged on a linear motor. In this way, the analysis system can be brought closer to a test strip for a more accurate analysis. This configuration is particularly interesting when the urine analysis device has two rotatable holders. This is because the analysis system can perform a conclusive analysis on a test strip stored in the radially inner rotatable holder 442.

The analysis system 50 may include a light source, such as one or more light emitting diodes (LEDs). The one or more light sources illuminate a test strip. To simplify the remainder of the description, the light source will be an LED.

Preferably, the analysis system 50 has multiple wavelength LEDs specific to the different reagents contained in different types of test strips. Thus, the urine analysis device can perform different analyses accurately.

Alternatively, the analysis system 50 may include a single LED. For example, the LED may be white. Then, the LED may cover the entire visible spectrum. This configuration reduces the complexity of the analysis system.

The analysis system 50 may also include a collimator. The collimator is used to direct the illumination of the LED(s) toward the test strip.

The analysis system 50 also includes the optical sensor 99. In particular, the optical sensor 99 is of the photodiode, CCD (“Charged Coupled Device”) or CMOS (“Complementary Metal Oxide Semiconductor”) type. The optical sensor can then measure the absorbance or fluorescence of the reagent in the test strip, particularly by transmission or reflection, to establish the analysis result(s).

The optical sensor 99 can be topped with a filter. The filter increases the sensitivity of the optical sensor to specific wavelengths. The accuracy of an analysis is then very satisfactory.

A particular embodiment of the analysis system 50, illustrated in FIG. 30, is described next. The analysis system 50 described below is particularly suited to the rotatable holder 44 storing the test strips 56 in an inner wall 44b of the rotatable holder 44.

Here, the analysis system 50 is arranged on either side of the rotatable holder 44 when inserted into the case 22. The analysis system 50 overlaps the rotatable holder 44 inserted into the case 22. A first portion 50a of the analysis system 50 is radially external to the rotatable holder 44 and a second portion 55b of the analysis system 50 is radially internal to the rotatable holder 44. The analysis system 50 operates by transmitting light from the first portion 55a to the second portion 55b.

The first portion 55a comprises at least one light source, for example in the form of a pair of LEDs 176, 178. Each LED in the pair is aligned with a hole 160 provided on the separator 158 of the rotatable holder 44. Light is guided through the holes 160 to illuminate the test strips 56. A first LED 176 of the pair is white in color to cover the entire visible spectrum and determine color changes of the test strip 56. A second LED 178 of the pair is monochromatic, such as ultraviolet, to excite fluorophores and allow observation of their emission wavelength.

The analysis system 50 could include one pair of LEDs or two pairs of adjacent LEDs. Depending on the number of pairs of LEDs, it appears possible to analyze multiple test strips 56 simultaneously.

The second portion 55b comprises the optical sensor 99. The optical sensor 99 is here of spectral type. It comprises several photodiodes topped by filters allowing to measure the intensity of the light at different wavelengths distributed in the visible range. The sensor 99 is compatible with optical measurements by absorbance and fluorescence.

The sensor 99 can be used for different types of test strips. In the example shown in FIG. 30, the test strip 56 is of the immuno-chromatographic type and has a test area and a control area aligned with the holes 160, respectively, the color change of which provides a result. In another example, the test strip 56 may be a colorimetric strip and have two separate test areas (for example, to simultaneously test pH and urine specific gravity) aligned with the holes 160, respectively.

It should be noted that other configurations with, for example, more than two holes 160 can be envisaged to increase, for example, the number of tests performed with the same strip.

Communication and System Aspects

The urine analysis device includes a communication module 41. The communication is wireless. The communication module 41 operates a local area network, such as Bluetooth, Bluetooth Low-Energy (BLE) or Wi-fi. The local network allows to preserve a battery 94 of the test assembly 24. Thus, the autonomy of the analysis device is increased.

Alternatively, the communication module 41 may operate a cellular telecommunications network. For example, the cellular communication network may be GSM, 3G, 4G, 5G, 4G-LTE. The communication module 41 then has a longer range.

Alternatively, the communication module 41 may operate a gateway connected to the cellular telecommunications network. In particular, the gateway may be a router, for example a Wi-fi router connected to the cellular network, a hub, i.e., a device connected directly to the cellular network, or the user's smartphone. Then the urine analysis device can be connected to the cellular network without compromising the battery life 94.

Preferably, the communication module 41 uses Bluetooth Low Energy (BLE) technology to communicate with a user's smartphone 61 and Wi-Fi technology to connect to a remote server 98.

The communication module 41 allows an analysis to be triggered via a remote control.

Preferably, the user can initiate an analysis directly from the smartphone 61. The user has control over the urine analysis device, and can choose when to perform an analysis, as well as the type of analysis they wish to perform. In addition, the user can be identified by the smartphone. The analysis performed can be tailored to the identified user, and the results sent to enrich the history of that identified user.

Alternatively, the analysis is initiated by communication with a remote device 42 near the toilet.

The remote device has a button 55. The user can then press the button to initiate an analysis. The user has control over the urine analysis device, and can choose when to perform an analysis.

The button 55 can be provided with a biometric sensor 57. The button can then identify the user pressing the button. Then, an analysis relevant to the identified user can be performed by the urine analysis device. Furthermore, the analysis performed can be selected based on the identified user, and the results sent to enrich the history of that identified user.

The remote device 42 also includes a display 59. For example, the display may consist of one or more colored light emitting diodes (LEDs). The display may also include a screen. The display can inform the user. For example, the user may be informed that a button press has been perceived, and/or that the user has been identified and/or that an analysis is about to be performed.

Alternatively, the remote device 42 may be a connected bracelet associated with the user. In this case, the user may be automatically detected when in the vicinity of the toilet. The user can also be identified by the connected bracelet. Thus, an analysis can be launched automatically, without any action from the user. Note that the connected bracelet can be a connected watch.

The communication module 41 also allows the communication of the test analysis(es). The test analysis(es) may be one or more of the following: fertility period, pregnancy, urinary tract infections, liver problems, kidney failure, uric acidosis, dehydration, heart disease and/or diabetes. The result(s) can also be an indicator of medication compliance.

The communication module 41 may send the test analysis(es) directly to the display 59 or the smartphone 61. The communication module may then be without a connection to the cellular telecommunications network. The test analysis(es) may be interpreted locally by the electronic control unit 45, or by the smartphone application. Thus, the operating costs of the urine analysis device are reduced.

Alternatively, the communication module 41 may send the one or more results to a remote server 98. The remote server may interpret the one or more test analyses. The use of a server reduces the computational capacity required locally to interpret the test analysis(es).

The remote server 98 may also be provided with storage capacity. Thus, the remote server may store the result(s) of a plurality of successive scans, for one (or each) user.

The user can view and evaluate one or more test analyses, received directly from the testing device or server saying. For example, the user can view and exploit the results from the smartphone application. Alternatively, the user can access a website from a computer, to access these result data.

In addition, the case 22 may directly present information to the user. The case 22 may present an indicator light 194, including one or more light emitting diodes (LEDs). The indicator light 194 may provide communication between the case 22 and the user. Different display colors can convey specific messages. For example, a red light may indicate a low battery level. The indicator light 194 may also indicate to the user that the case 22 is correctly positioned on the mounting element 61.

Finally, a button 192 may be accessible to the user from the case 22. The button 192 is accessible to the user to reset the urine analysis device 12. In particular, the button 192 may be accessible to the user when the removable cover 90 is removed.

Control Unit

The electronic control unit 45 may be divided into a plurality of sub-circuits (see, for example, FIG. 31). The sub-circuits allow flexibility in their arrangement within the case 22 of the analysis device.

A main circuit 180 may include a main microcontroller. The main circuit 180 may control the motors 76, 91, the pump 86, and the communication module 41. The main circuit 180 is also capable of cooperating with the other circuits to enable coordination of the other circuits.

An optical analysis circuit 182 may cooperate with the optical sensor 99 of the analysis system 50. The optical analysis circuit 182 may be connected to a first diode circuit 184. The first diode circuit 184 may control the LED(s) of the analysis system 50 to colorimetrically analyze the test strips 56.

An indicator light circuit 186 can control the indicator light 194 for communication with the user from the case 22.

A volume and flow rate sensing circuit 188 may include circuit board 174 having electrodes e1, e2, 23. The volume and flow rate sensing circuit 188 is responsive to signals from the electrodes when in contact with urine.

An ancillary circuit 196 may provide access to a test and diagnostic interface, allowing quality testing of the urine analysis device 12. The ancillary circuit 196 may be connected to a reset circuit 190 comprising the reset button 192 for the urine analysis device 12.

Specifically, the main circuit comprises a first Bluetooth Low Energy System on Chip (SOC). The first system-on-chip enables communication with the smartphone 61 and manages the other circuits. The first system-on-chip cooperates with a second Wifi system-on-chip (812.11) that handles data exchange with the server 98. This architecture optimizes the consumption of the battery 94 by keeping the second system-on-chip, which consumes a lot of energy, switched off when it is not in use.

The electronic control unit 45 is powered by the battery 94 provided inside the case 22. The battery 94 is of the lithium-ion type. The capacity of the battery is approximately 1080 mAh. Such a capacity makes it possible to ensure a satisfactory autonomy of the device without compromising the dimensions of the case.

The battery 94 comprises charging connectors. The charging connectors are accessible from the exterior of the case 22 to allow charging of the battery 94. For example, the case 22 may be placed on a stand to connect the charging connectors to a power source. Alternatively, charging by induction could also be considered.

Process Aspects

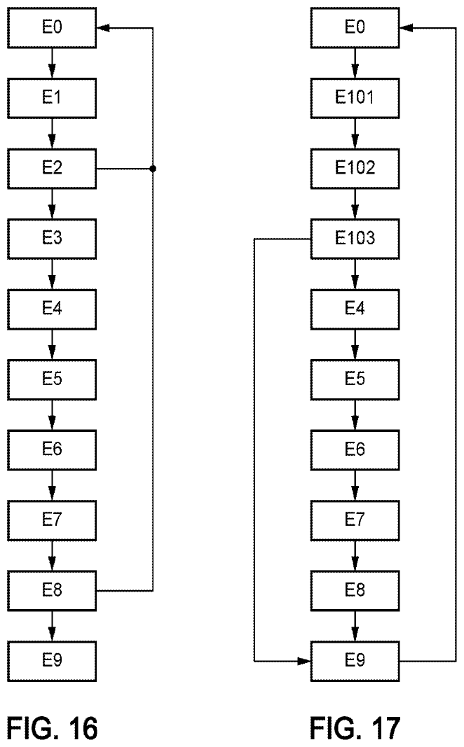

Hereinafter, two processes for performing an analysis using the above-described urine analysis device are described in more detail, with reference to FIG. 16 and FIG. 17. The processes are performed by the electronic control unit 45. As shown in FIG. 15, the electronic control unit 45 receives information from the sensors 38, 99 and controls the motors 76, 91 and the pump 86.

The first process, illustrated in FIG. 16, consists of launching an analysis following a request from the user.

Beforehand, it is noted that the case 22 is positioned in the toilet. The rotatable holder 44 has at least one unused test strip 56 and the device battery 98 is sufficiently charged. If this is not the case, the device indicates to the user that the test cannot be performed.

Step E0 is to hold the urine analysis device in a purge position. The opening 74 of the rotatable holder is aligned with the automated syringe 80 of the injector 46. The automated syringe passes through the opening of the rotatable holder. The automated syringe is inserted into the purge channel or directly accesses the drain port 34. Thus, urine or water received from the toilet in the collection channel 82 can reach the drain port 34.

In step E1, the urine analysis device receives the urine analysis request. The analysis request may come from the user pressing the button 55. The analysis request may also be ordered from the smartphone 61. The analysis request may also be performed automatically, when the user is in the vicinity of the toilet. The display 59 may indicate to the user that their press has been received. The display can also indicate whether a user was recognized when the button was pressed.

In step E2, the urine analysis device waits to detect urine near the collection port. The case may further be equipped with LEDs to alert a user that the urine analysis device is waiting to detect urine. The LEDs can be those of the indicator light 194.

If no urine stream is received within a specified time, then the urine analysis device returns to step E0.

If, instead, a stream of urine is detected, step E3 activates the pump 86 to deliver urine from the collection port to the automated syringe 80. Step E3 stops when enough urine is detected in the internal buffer space or in the automated syringe to perform an analysis. If the injector 46 comprises the urine monitoring means 168, step E3 stops when the reference volume bounded by the electrodes e1 and e2 is detected in the cavity 172 of the sleeve 170.

In addition, when the injector 46 comprises the urine monitoring means 168, step E3 comprises calculating the flow rate of urine flowing between the electrodes e1 and e3. The urine flow rate is used to determine the pump activation time required to inject a specific volume.

In addition, when the injector 46 comprises the urine control means, step E3 may include reactivating the pump to expel the pre-injection volume to the drain port 34. The needle 96 is then devoid of air, which due to its compressibility may impact the pump activation time required to inject the precise volume.

Step E4 involves positioning the urine analysis device in a pre-injection position. The pre-injection position prepares the urine analysis device for an injection of urine onto a test strip. The automated syringe 80 is retracted from the purge channel or drain port 34 and the opening 74 of the rotatable holder, or was already in a retracted position. The rotatable holder is rotated so that a housing on the rotatable holder faces the automated syringe.

Note that steps E3 and E4 can be performed simultaneously.

Step E5 then consists in positioning the urine analysis device into an injection position. The automated syringe 80 is translated through the action of motor 91, to pierce the lid 72. The automated syringe injects urine onto a test strip. The injected urine can then react with the reagents on the test strip. After the actual injection, the needle can be retracted.

The injection of urine in step E5 can be done in two steps. For example, the automated syringe 80 can inject between 2.5 microliters and 3.5 microliters twice. This solution allows for a reaction and migration time of the urine on the test strip 56.

When the urine analysis device comprises the urine monitoring means 168, the injection of urine can be performed by activating the pump for the time determined in step E3. This ensures that an accurate and repeatable volume is injected onto the test strip 56.

Step E6 involves positioning the urine analysis device in the analysis position. First, it is verified that the automated syringe is retracted from the housing. The automated syringe returns to the pre-injection position. The rotatable holder rotates to position the test strip that received the urine in step E4 facing the analysis system.

Step E7 is to establish the analysis result(s). The analysis system 50 performs a colorimetric analysis on the test strip. The urine analysis device derives the analysis result(s). The analysis performed depends on the type of test strip. The analysis performed may also depend on a choice of the user. The analysis performed can also be selected based on the identified user.

Step E8 corresponds to the transmission of the result(s). The result(s) can be transmitted directly to the user. The result(s) may also be sent to the server 98. The user may, for example, view and evaluate the result(s) on the smartphone application 61, or on a website. The result(s) may also be sent to a healthcare professional.

Step E9 involves purging the urine analysis device. The pump 86 is activated to push air into the delivery means 48. The urine is then expelled from the urine analysis device via the purge channel and through the drain port 34. The urine analysis device then returns to the purging position of step E0.

Note that steps E4 to E7 can be repeated several times. Thus, several test strips receive urine and are analyzed. Thus, several analyses can be performed from a single urine aspiration in step E3.

Alternatively, in the example shown in FIG. 17, the process is initiated when urine is detected on the case.

Step E0 consists of holding the urine analysis device in the purge position, as described above.

Step E101 is to detect the presence of urine in the vicinity of the collection port 32.

Then, step E102 is to activate the pump 86 to draw urine from the collection port 32 to the automated syringe 80. Step E3 stops when enough urine is detected in the automated syringe and/or the internal buffer to perform an analysis. Step E3 may also stop when sufficient urine is detected by the urine monitoring means 168. Step E3 may also include determining the urine flow rate and reactivating the pump as described above. The display 59 may indicate that an analysis is ready to be performed. Alternatively, the case, provided with LEDs, may indicate that an analysis is ready to be performed.

In step E103, the urine analysis device waits for receipt of an analysis request from a user. The analysis request may originate from the user pressing the button 55. The analysis request may also be commanded from the smartphone 61.

If the analysis request is not received within a specified time period, then the pump is activated to purge the urine analysis device of the collected urine (Step E9). The urine analysis device returns to the purge position of step E0.

If instead the analysis request is received, then an analysis is performed. The urine analysis device then performs steps E4 to E9 as described above.

1. A urine analysis device to be positioned within a toilet comprising a test assembly, which comprises:
a rotatable holder comprising a plurality of test strips attached to the rotatable holder;
an injector configured to inject a controlled volume of urine onto at least one of the plurality of test strips;
an analysis system configured to detect a result of urine injection onto the at least one of the plurality of test strips.

2. The urine analysis device according to claim 1, wherein the rotatable holder is configured to rotate a test strip and selectively present said test strip in front of the injector and/or the analysis system.

3. (canceled)

4. The urine analysis device according to claim 1, wherein each the rotatable holder has housings receiving one or more test strips.

5. The urine analysis device according to claim 1, wherein the housings are closed by at least one lid.

6. The urine analysis device according to claim 4 or 5, wherein the rotatable holder is cylindrical, and the housings are arranged on an outer wall of the rotatable holder.

7. The urine analysis device according to claim 4, wherein the rotatable holder is cylindrical, and the housings are arranged on an inner wall of the rotatable holder.

8. The urine analysis device according to claim 4, wherein the rotatable holder comprises a separator comprising the housings, the separator being made of a flexible material, preferably elastomer, and a receptacle receiving the separator.

9. The urine analysis device according to claim 8, wherein the receptacle comprises an annular portion and a cylindrical portion, radially external to the annular portion.

10. The urine analysis device according to claim 9, wherein the separator comprises holes opening into the housings, and the cylindrical portion contacting the separator is transparent.

11. The urine analysis device according to claim 1, wherein the plurality of test strips are distributed along a circle or a circular arc about an axis of rotation of the rotatable holder.

12. The urine analysis device according to claim 1, wherein the injector comprises a urine monitoring apparatus, the urine monitoring apparatus comprising:
a sleeve defining an internal cavity through which urine can flow;
a first conductive electrode and a second conductive electrode extending through an outer wall of the sleeve to open into the cavity so as to be responsive to urine received in the cavity, the first electrode being spaced from the second electrode to delineate a reference volume of the cavity.

13. The urine analysis device according to claim 12, wherein the urine monitoring apparatus comprises a third conductive electrode, disposed between an end of the sleeve and the first conductive electrode for measuring a flow rate of urine flowing through the cavity.

14. The urine analysis device according to claim 1, wherein the analysis system comprises:
at least one light source disposed on one side of the rotatable holder, the at least one light source emitting light toward the rotatable holder, and, a sensor disposed on the other side of the rotatable holder, the sensor facing the at least one light source so as to detect light passing through the rotatable holder.

15. The urine analysis device according to claim 1, wherein a case encloses the test assembly, the case being configured to be positioned entirely within a toilet.

16. The urine analysis device according to claim 1, wherein the rotatable holder is removably arranged in the case.

17. The urine analysis device according to claim 1, wherein the test assembly further comprises a urine presence sensor, the sensor being for example a temperature sensor.

18. (canceled)

19. The urine analysis device according to claim 1, wherein the injector is arranged in a radially inner area of the rotatable holder.

20. The urine analysis device according to claim 1, wherein the injector comprises a movable syringe configured to be moved toward or away from a test strip.

21. (canceled)

22. A method of urine analysis using the urine analysis device according to claim 1, comprising:
positioning a test strip in front of the injector by rotation of the rotatable holder;
injecting a controlled volume of urine onto the test strip, and
analyzing the test strip to establish an analysis result.

23. The urine analysis device according to claim 13, wherein the sensor is configured to control an angular position of the rotatable support by detecting one or more markers on the rotatable holder.

24.-58. (canceled)
